(19) United States
(12) Patent Application Publication
(10) Pub. No.: US 2026/0364108 A1
(43) Pub. Date: Nov. 19, 2026

(54) FGFR4 ANTIBODY CONSTRUCTS AND THEIR USE

(71) Applicant: The Chinese University of Hong Kong, Shatin (CN)

(72) Inventors: Nathalie WONG, Fo Tan (CN); Mingjing XU, Tin Hau (CN)

(21) Appl. No.: 19/196,026

(22) Filed: May 1, 2025

(51) Int. Cl. A61K 35/17; A61K 9/00; A61K 40/13; A61K 40/31; A61K 40/42; A61K 45/06; A61K 47/68; A61K 48/00; A61P 35/00; C07K 14/705; C07K 16/28; C12N 5/0783; C12N 15/86; C12N 15/88

(52) U.S. Cl. CPC A61K 35/17; A61K 9/0019; A61K 40/13; A61K 40/31; A61K 40/4206; A61K 45/06; A61K 47/6849; A61K 48/005; A61P 35/00; C07K 14/7051; C07K 14/70521; C07K 16/2863; C12N 5/0636; C12N 15/86; C12N 15/88; A61K 2239/13; A61K 2239/21; A61K 2239/31; A61K 2239/53; C07K 2317/565; C07K 2317/622; C12N 2740/15043

(57) ABSTRACT

This disclosure relates to novel antibody constructs that specifically bind one variant of the fibroblast growth factor receptor 4 (FGFR4) protein, FGFR4-Δ, which is preferentially expressed in certain cell types, especially malignant cell types. Such antibody constructs include a single-chain variable fragment (scFv) derived from an anti-FGFR4-Δ monoclonal antibody as well as a chimeric antigen receptor (CAR) construct generated by incorporating the scFv. The present invention also provides methods for diagnosing and treating conditions including cancer characterized by the expression of FGFR4-Δ in the pertinent tissue and cells through the use of these anti-FGFR4-Δ antibody constructs.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Sep. 11, 2025, is entitled “080015-1498097-043000US_ST26.xml”, and is 64,744 bytes in size.

### BACKGROUND OF THE INVENTION

Hepatocellular carcinoma (HCC) is the most common type of primary liver cancer and is the leading cause of cancer-related deaths worldwide. Despite advancements in diagnostic techniques and treatment options, most HCC cases are diagnosed at advanced stages, resulting in limited therapeutic options. Although antibody-based therapies and adoptive cell transfer (ACT) approaches have transformed cancer treatment by targeting tumor-associated antigens, their effectiveness in treating solid tumors, such as HCC, remains unsatisfactory due to the lack of specific antigens.

Perturbed alternative splicing (AS) contributes to the generation of cell type-specific membrane proteins and has been shown to play critical roles in cancer progression. To identify HCC-specific antigens, long-read RNA sequencing was deployed on patient-derived HCC organoids and identified an unannotated spliced variant of fibroblast growth factor receptor 4 (FGFR4), referred to as FGFR4-Δ. Expression of FGFR4-Δ was specifically found in HCC tissues and was associated with inferior survival outcomes of HCC patients. Functional studies revealed that FGFR4-ΔEx3 enhances cell viability, colony formation, and migration in HCC cells via sustaining persistent oncogenic signaling.

In an effort to specifically target FGFR4-Δ, the present inventors developed several FGFR4-Δ-specific monoclonal antibodies. ELISA and flow cytometry assays confirmed their specific binding to FGFR4-Δ with minimal cross-reactivity to the canonical FGFR4-full length (FL) variant. The single-chain variable fragment (scFv) derived from these antibodies was used to generate FGFR4-Δ-specific CAR-T cells. Engineered CAR-T cells exhibited strong specificity and potent cytotoxicity against FGFR4-Δ expressing cells in vitro. In vivo studies further validated the tumor-suppressive effect of FGFR4-Δ CAR-T therapy in subcutaneous xenograft models.

This study establishes FGFR4-Δ as a novel tumor-specific antigen in HCC with strong oncogenic properties. Targeting FGFR4-Δ with monoclonal antibodies or CAR-T therapy presents promising therapeutic strategies for HCC.

### BRIEF SUMMARY OF THE INVENTION

This disclosure provides a previously unknown FGFR4 variant, termed FGFR4-Δ, monoclonal antibodies that specifically binds to FGFR4-Δ, including a single-chain variable fragment (scFv) derived from an anti-FGFR4-Δ monoclonal antibody. Also provided is a chimeric antigen receptor (CAR) construct generated from the anti-FGFR4-Δ scFv, which may further include a biologically-active molecule in order to direct T cells to target FGFR4-Δ-expressing cancer cells.

As such, in a first aspect, this invention provides an anti-FGFR4-Δ antibody construct that specifically binds FGFR4-Δ and comprises antibody light chain and heavy chain complementarity determining regions (CDRs) having the amino acid sequences listed in Table 1 and Table 2. The anti-FGFR4-Δ antibody construct of this invention may be a polypeptide in the entirety, or it may be a polypeptide with at least one non-polypeptide component conjugated to it.

In some embodiments, the FGFR4-Δ antibody construct of this invention comprises an antibody light chain variable region (V_{L}) and an antibody heavy chain variable region (V_{H}), each comprising the amino acid sequence set forth in SEQ ID NOs: 12-22 and 1-11, respectively. In some embodiments, the FGFR4-Δ antibody construct is an anti-FGFR4-Δ single chain variable fragment (scFv), for example, it specifically binds FGFR4-Δ and comprises an antibody light chain and heavy chain variable regions comprising the CDRs having the amino acid sequence set forth in SEQ ID NOs: 23-33, respectively. In some embodiments, the anti-FGFR4-Δ scFv comprises the amino acid sequence of SEQ ID NO:23. In some embodiments, the FGFR4-Δ antibody construct is a chimeric antigen receptor (CAR) comprising, from its N-terminus, a FGFR4-Δ scFv, a CD28 transmembrane domain, and a CD3ζT cell activating domain. For example, the CAR construct comprises the amino acid sequences set forth in SEQ ID NO:23, SEQ ID NO:36, and SEQ ID NO:37.

In a second aspect, the present invention provides a nucleic acid comprising a polynucleotide sequence encoding the anti-FGFR4-Δ antibody construct, a fusion polypeptide described above and herein, for example, an anti-FGFR4-Δ scFv or anti-FGFR4-Δ CAR construct. The nucleic acid may comprise an expression cassette comprising a promoter operably linked to the polynucleotide sequence encoding the anti-FGFR4-Δ antibody construct. In some cases, the nucleic acid is in the form of a vector, e.g., a replicable vector such as an expression vector including a plasmid or a viral vector.

In some embodiments, the nucleic acid is contained and present within a host cell, either in the form of a free vector or in the form of DNA sequence permanently integrated into the host cell genome. The presence of such nucleic acid ensures the expression of the anti-FGFR4-Δ polypeptide antibody construct of this invention, e.g., an anti-FGFR4-Δ polypeptide scFv or anti-FGFR4-Δ CAR construct. Exemplary amino acid sequences of such scFv constructs include SEQ ID NOs: 23-33. In some embodiments, the host cell is a T cell or natural killer (NK) cell. In some embodiments, the host cell is a T cell expressing a CAR comprising the amino acid sequences set forth in SEQ ID NOs: 23, 36, and 37.

In a third aspect, the present invention provides a conjugate comprising the FGFR4-Δ polypeptide antibody construct of the present invention. The conjugate includes a polypeptide component, i.e., the anti-FGFR4-Δ polypeptide antibody fusion polypeptide of this invention, and one or more conjugation partners, which may or may not be a protein in nature. For example, the conjugation partner may be a solid support, a detectable moiety, or a therapeutic agent. In some embodiments, the conjugate includes an anti-FGFR4-Δ polypeptide antibody construct, such as an anti-FGFR4-Δ polypeptide scFv, and a therapeutic agent, such as an anti-cancer therapeutic agent. In this connection, compositions are also provided, which comprise the FGFR4-Δ polypeptide antibody construct of this invention, its encoding nucleic acid, including in the form of an expression cassette or vector, a host cell containing the nucleic acid and/or expressing the anti-FGFR4-Δ polypeptide antibody construct, or the conjugate comprising the anti-FGFR4-Δ polypeptide antibody fusion polypeptide of this invention joined with a conjugation partner, which may be another polypeptide or of a non-protein chemical nature.

In a fourth aspect, the present invention provides a method for killing FGFR4-Δ polypeptide-expressing cells. The method includes a step of contacting FGFR4-Δ polypeptide-expressing cells, e.g., FGFR4-Δ polypeptide-positive cancer cells, with a composition comprising an effective amount of an anti-FGFR4-Δ polypeptide antibody construct, preferably conjugated with a therapeutic agent, e.g., anti-cancer agent, or an adequate number of T cells expressing the FGFR4-Δ polypeptide antibody CAR construct described above and herein, e.g., comprising the amino acid sequences of SEQ ID NOs: 23, 36, and 37.

In some embodiments, the FGFR4-Δ polypeptide+ cells being targeted by the claimed methods are cancer cells characterized by their FGFR4-Δ polypeptide expression, for example, hepatocellular carcinoma (HCC) cells. In some embodiments, the cancer cells are located in a patient's body. In some embodiments, the method further include these steps, prior to the step of contacting FGFR4-Δ polypeptide-expressing cells (e.g., FGFR4-Δ polypeptide-positive cancer cells) with a composition comprising an adequate number of T cells expressing the FGFR4-Δ polypeptide antibody CAR construct, (i) isolating T cells from a patient; (ii) transfecting the T cells with the nucleic acid encoding the anti-FGFR4-Δ polypeptide CAR construct; (iii) cultivating the T cells ex vivo to expanding T cells expressing the anti-FGFR4-Δ polypeptide CAR construct comprising, from its N-terminus, an anti-FGFR4-Δ polypeptide scFv, a CD28 transmembrane domain, and a CD3ζT cell activating domain, and (iv) administering the expanded T cells in sufficient number back into the patient's body. Exemplary CAR constructs comprise the amino acid sequence set forth in SEQ ID NOs: 23, 36, and 37. In some embodiments, the administering step comprises injection, e.g., by subcutaneous, intravenous, intramuscular, intraperitoneal, or intratumoral injection.

In a fifth aspect, the present invention provides a method for treating a disease involving FGFR4-Δ expression in a patient. The method includes a step of administering to the patient with a composition comprising an effective amount of a nucleic acid encoding an anti-FGFR4-Δ CAR construct described above and herein. In some embodiments, the disease is a cancer, such as hepatocellular carcinoma (HCC). In some embodiments, the nucleic acid encoding the CAR construct is a viral vector. In some embodiments, the composition comprises the nucleic acid formulated with lipid nanoparticles. In some embodiments, the CAR construct comprises the amino acid sequences set forth in SEQ ID NOs: 23, 36, and 37. In some embodiments, the administering step comprises subcutaneous, intravenous, intramuscular, intraperitoneal, or intratumoral injection.

In a sixth aspect, the present invention provides a kit for treating a disease or condition characterized by FGFR4-Δ polypeptide expression, for example, FGFR4-Δ polypeptide+ cancers. The kit often includes a first container containing a first composition comprising an adequate number of T cells expressing a CAR comprising, from its N-terminus, an anti-FGFR4-Δ scFv, a CD28 transmembrane domain, and a CD3ζ T cell activating domain, and a second container containing a second composition comprising an effective amount of another therapeutic agent known for its efficacy for treating the disease or condition, such as an anti-cancer agent. In some embodiments, the FGFR4-Δ polypeptide+ cancer is hepatocellular carcinoma. In some embodiments, the anti-FGFR4-Δ polypeptide CAR comprises the amino acid sequences set forth in SEQ ID NO:23, 36, and 37. In some embodiments, the first or second composition is formulated for injection, e.g., for subcutaneous, intravenous, intramuscular, intraperitoneal, or intratumoral injection. In some cases, the kit may further comprise an instruction manual providing instructions for user to properly use the kit.

Lastly, the present invention discloses for the first time FGFR4-Δ, a novel FGFR4 variant, due to alternative splicing. The amino acid sequence for this previously unknown variant is set forth in SEQ ID NO:41. Thus, the present invention provides a polypeptide comprising the amino acid sequence set forth in SEQ ID NO:41. In some embodiments, the polypeptide includes the amino acid sequence set forth in SEQ ID NO:41 conjugated with a heterologous moiety, which may be of any chemical/structural characteristics and is useful for detection, purification, etc. In some embodiments, the polypeptide is a fusion polypeptide comprising the amino acid sequence of SEQ ID NO:41 and a peptide tag heterologous to SEQ ID NO:41. Also provided includes a nucleic acid, a host cell, and a composition related to the FGFR4-Δ variant: (1) a nucleic acid comprising a polynucleotide sequence encoding a polypeptide comprising the amino acid sequence set forth in SEQ ID NO:41. The nucleic acid may optionally further comprise a nucleotide sequence that is heterologous to FGFR4-Δ coding sequence. The nucleic acid may be in the form of an expression cassette comprising a promoter (e.g., a heterologous promoter) operably linked to the polynucleotide sequence encoding FGFR4-Δ polypeptide or in the form of vector (e.g., a plasmid or a viral vector). (2) A host cell comprising the nucleic acid described above or herein (encoding a polypeptide) or the polypeptide comprising the amino acid sequence of SEQ ID NO:41. (3) A composition comprising the FGFR4-Δ polypeptide or the FGFR4-Δ-encoding nucleic acid described above and herein, or the host cell containing the FGFR4-Δ-encoding nucleic acid or FGFR4-Δ polypeptide, typically with one or more physiologically acceptable excipients. In some embodiments of the inventive aspects described above and herein, the polynucleotide sequence encoding a FGFR4-Δ polypeptide is set forth in SEQ ID NO:42.

### BRIEF DESCRIPTION OF THE DRAWINGS

FIG. 1. FGFR4-Δ was identified in HCC. (A) Schematic representation of FGFR4-Full length (FL) and FGFR4-Δ variants shown with exon arrangement and AS sites highlighted (dotted rectangle). Sanger sequencings confirmed unannotated junctions of FGFR4-Δ in HCC tumor specimen. (B) AlphaFold predicted protein structure of FGFR4-FL and FGFR4-Δ. Structural alteration in the extracellular domain due to exon skipping are highlighted (dotted circles). (C) Immunoprecipitation result revealed FGFR4-FL and FGFR4-Δ as two dominant FGFR4 isoforms in HCC. (D) Immunoblotting showed FGFR4-Δ was specifically expressed in HCC tumor (T) tissues, whereas FGFR4-FL can be seen in both the tumor (T) and adjacent non-tumor (NT) liver tissues. (E) Taqman probe-based qPCR analysis demonstrated a significant upregulation of FGFR4-Δ in HCC tumor (T) compared to adjacent non-tumor (NT) and normal liver (NL) tissues. (F) FGFR4-Δ upregulation was detected in over 63.5% of HCC cases. A fold Change (FC) of tumor versus non-tumor greater than 4 was used as the cutoff. (G) Kaplan-Meier analysis showed that high expression of FGFR4-Δ significantly correlated with shorter overall and disease-free survival in HCC. **P≤0.01, ****P≤0.0001 by Student's t-test.

FIG. 2. FGFR4-Δ exhibited oncogenic effects in HCC. (A) Representative immunofluorescence images showing that FGFR4-Δ localization at the plasma membrane. HKCI-2 cells overexpressing flag-tagged FGFR4-Δ were stained with anti-flag antibody. (B-D) Overexpression of FGFR4-Δ promoted viability (B), colony formation (C), and migration (D) in HKCI-2 cells. (E-F) Ectopic expression of FGFR4-Δ following FGFR4 knockout enhanced viability of HCC tumor organoid (HCC.Org_813T) and Hep3B cells. (G-H) Overexpression of FGFR4-Δ following FGFR4 knockout promoted colony formation (G) and migration (H) in Hep3B cells. (I) Presence of FGFR4-Δ led to stronger and more sustained FGFR4 phosphorylation upon FGF19 treatment. Cells were treated with FGF19 and harvested at the indicated time points. Immunoblot intensities were quantified and shown. ns: not significant, *P≤0.05, **P≤0.01, ****P≤0.0001 by Student's t-test (C, D, G, and H) or two-way ANOVA (B, E, and F).

FIG. 3. FGFR4-Δ specific mouse monoclonal antibodies were screened and validated. (A) ELISA results showing that 11 mouse monoclonal antibodies were specifically recognize FGFR4-Δ, but not FGFR4-FL. (B) Flow cytometry analysis confirmed the specific binding of anti-FGFR4-Δ antibody (S3-357-7) to FGFR4-Δ protein. HKCI-2 cells expressing empty vector (EV), FGFR4-FL or FGFR4-Δ were incubated with anti-FGFR4-Δ S3-357-7 antibody for 30 minutes on ice, followed by incubation with APC-conjugated secondary antibody to detect primary antibody binding. (C) Summary of flow cytometry analysis for 11 anti-FGFR4-Δ antibodies showing their binding specificity to FGFR4-Δ.

FIG. 4. FGFR4-Δ specific antibody recognizes endogenous FGFR4-Δ in HCC cell lines. (A) qPCR results showing the differing levels of FGFR4-Δ expression in HCC cell lines. (B) Flow cytometry confirmed that the FGFR4-Δ-specific antibody (S3-357-7) bound to HCC cell lines in accordance with FGFR4-Δ mRNA expression, while no binding is observed in FGFR4-knockout (KO) Hep3B cells.

FIG. 5. FGFR4-Δ CAR-T cells exhibited anti-tumor cytotoxicity in vivo and in vitro. (A) CAR FGFR4-Δ construct was transduced into primary human T cells. GFP positive cells indicated successful transduction. (B) Anti-FGFR4-Δ CAR-T cells efficiently lyse HepG2 cells overexpressing FGFR4-Δ (HepG2 FGFR4-ΔOE), while exhibiting minimal effect on HepG2 empty vector cells, which have low FGFR4-Δ expression. (C) Anti-FGFR4-Δ CAR-T cells demonstrated strong cytotoxic effects in subcutaneous xenografts derived from HepG2 FGFR4-Δ^{OF} cells compared to mock CAR-T cells. ****P≤0.0001 by two-way ANOVA.

### DEFINITIONS

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

An “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to the variable region of the light and heavy chains, respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, 3^{rd} Edition, Raven Press, NY (1993)). While various antibody fragments are defined in terms of the digestion of an intact antibody, one of skill will appreciate that such fragments may be synthesized de novo either chemically or by utilizing recombinant DNA methodology.

Further modification of antibodies by recombinant technologies is also well known in the art. For instance, chimeric antibodies combine the antigen binding regions (variable regions) of an antibody from one animal with the constant regions of an antibody from another animal. Generally, the antigen binding regions are derived from a non-human animal, while the constant regions are drawn from human antibodies. The presence of the human constant regions reduces the likelihood that the antibody will be rejected as foreign by a human recipient. On the other hand, “humanized” antibodies combine an even smaller portion of the non-human antibody with human components. Generally, a humanized antibody comprises the hypervariable regions, or complementarity determining regions (CDR), of a non-human antibody grafted onto the appropriate framework regions of a human antibody. Antigen binding sites may be wild type or modified by one or more amino acid substitutions, e.g., modified to resemble human immunoglobulin more closely. Both chimeric and humanized antibodies are made using recombinant techniques, which are well-known in the art (see, e.g., Jones et al. (1986) Nature 321:522-525).

Thus, the term “antibody,” as used herein, also includes antibody fragments either produced by the modification of whole antibodies or antibodies synthesized de novo using recombinant DNA methodologies (e.g., a Fab′, a F(ab)′₂, a single chain fragment variable or scFv, a chimeric or humanized antibody, and a chimeric antigen receptor or CAR) that retain the ability to specifically bind the same intended target antigen. For example, an scFv is composed of a V_{H} and a V_{L} connected by a peptide linker of a relatively short length (e.g., up to 25 amino acids), whereas a CAR is a recombinant fusion protein composed of an antigen-binding domain (typically an scFv) linked to (typically via a peptide linker) one or more signaling domains for activating immune cells (e.g., T cells or NK cells).

As used herein, a “chimeric antigen receptor” or “CAR” describes a single chain polypeptide comprising at least three domains: an extracellular antigen-binding ectodomain, a transmembrane domain, and an intracellular endodomain. The extracellular ectodomain includes at least a portion of a heavy chain variable region (V_{H}) and at least a portion of a light chain variable region (V_{L}) of an antibody (e.g., a single chain antibody or scFv). The intracellular endodomain acts to transmit intracellular signals triggered by the binding of an antigen (e.g., FGFR4-Δ) to the extracellular ectodomain. Thus, the CAR endodomain contains at least one signaling domain capable of activating an effector cell, for example, a T cell. As CAR constructs are designed for use in T cells, they are also termed chimeric T cell receptor. The term CAR T is often used to collectively refer to a CAR construct (polypeptide) and T cells expressing the construct. For a review of the CAR T construct design and evolution, see Enblad et al., Human Gene Therapy 26 (8):498-505, 2015. In the first generation CAR constructs, the intracellular signaling domain contains only the CD3ζ chain of a T cell receptor (TCR) complex, whereas the second and third generation CAR constructs include one and two (respectively) costimulatory domains, e.g., CD28, 4-1BB, in addition to CD3ζ. Thus, CAR constructs have one effector cell (e.g., T cell) signaling domain, one transmembrane domain (e.g., CD28 transmembrane domain), and at least one signaling domains (e.g., CD3ζ signaling domain). “Chimeric antigen receptor (CAR) therapy” refers to the use of CAR constructs for therapeutic purposes, including for adoptive cell therapy, a therapeutic approach that typically includes first isolation from a patient and ex vivo expansion and/or manipulation of immune effector cells (e.g., NK cells or T cells) prior to eventual re-infusion of these cells back to the same patient. One example of such use is for the treatment of cancer. Cells used in CAR therapy are typically autologous (taken from the same individual patient and then put back to the same person) but can be allogeneic (taken from one individual and put into another, different individual of the same species, e.g., both humans, especially with similar genetic background) under some circumstances. Cells may be manipulated to express CAR constructs using any one of the well-known methodologies in the art, for example, transformation by a nucleic acid (in the form or DNA or RNA), transfection by a viral vector, electroporation, etc.

As used herein, the term “complementarity determining region” or “CDR” refers to three relatively short stretches of amino acid sequences located within each of an antibody variable regions of the heavy chain and the light chain (V_{H} and V_{L}). The three CDRs in each of the V_{H} and V_{L} are designated CDR1, CDR2 and CDR3, from the N-terminus to the C-terminus, with a full set of CDRs encompassing all six CDRs for both heavy and light chains. The amino acid sequences of the CDRs and framework regions can be determined using various well-known numbering systems in the art, e.g., Kabat, Chothia, International ImMunoGeneTics database (IMGT), Martin (updated Chothia), or Honneger's numbering scheme (Aho) (see, e.g., Chothia & Lesk, 1987, J. Mol. Biol. 196, 901-917; Chothia C. et al., 1989, Nature 342, 877-883; Chothia C. et al., 1992, J. Mol. Biol. 227, 799-817; Al-Lazikani et al., J. Mol. Biol 1997, 273 (4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29 (1):207-9 (2001); MacCallum et al., Antibody-antigen interactions: Contact analysis and binding site topography, J. Mol. Biol., 262 (5), 732-745 (1996); and Martin et al., Proc. Natl Acad. Sci. USA, 86, 9268-9272 (1989); Martin, et al., Methods Enzymol., 203, 121-153, (1991); Pedersen et al., Immunomethods, 1, 126, (1992); and Rees et al., In Sternberg M. J. E. (ed.), Protein Structure Prediction. Oxford University Press, Oxford, 141-172 1996, Honegger & Pluckthun, Yet Another Numbering Scheme for Immunoglobulin Variable Domains: An Automatic Modeling and Analysis Tool. JMB (2001) 309.3:657-670). Reference to CDRs as determined by Kabat numbering are based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk, J. Mol. Biol. 196:901-917 (1987)).

In contrast to the CDRs, “framework” or “framework region” refers to the amino acid sequences within a V_{H} or V_{L} other than the CDRs. The presence of three CDRs on each of the heavy and light chains divides the framework regions into four sub-regions (FR1, FR2, FR3, and FR4) on each of V_{H} and V_{L}, with CDR1 positioned between FR1 and FR2, CDR2 between FR2 and FR3, and CDR3 between FR3 and FR4. When used collectively and not specifying any particular sub-regions as FR1, FR2, FR3, or FR4, the term “framework” or “framework region” is used to refer to the entirety of all FRs together within the variable region of each or both of the heavy chain and light chain.

The term “immunoassay” describes an assay that uses an antibody to specifically bind an antigen. The immunoassay is characterized by the use of specific binding properties of a particular antibody to identify, isolate, target, and/or detect the presence or quantity of the antigen.

The phrase “specifically binds,” when used to describe the binding relationship between an antibody and its target antigen, refers to a binding reaction that is determinative of the presence of the antigen (e.g., FGFR4-Δ) in a heterogeneous population of proteins and other biologics. Thus, under designated immunoassay conditions, the specified antibodies bind to a particular polypeptide at least two times the background and do not substantially bind in a significant amount to other polypeptides or other antigens (e.g., other variants of FGFR4, including its full-length (FL) version) present in the sample. Specific binding to an antibody under such conditions may require an antibody that is selected for its specificity for a particular protein. For example, antibodies raised to a FGFR4-Δ protein can be selected to obtain only those antibodies that are specifically immunoreactive with that specific protein FGFR4-Δ and not with other proteins including related proteins, e.g., other variants of the FGFR4 protein, especially FGFR4-FL. This selection may be achieved by subtracting out antibodies that cross-react with molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically, a specific binding reaction (e.g., between the target antigen FGFR4-Δ and a FGFR4-Δ-specific antibody) will yield at least twice of the background signal or noise (e.g., between non target antigen such as FGFR4-FL and the FGFR4-Δ-specific antibody) and more typically more than 5, 10, 20, 50, or up to 100 times the background.

A “label,” “detectable label,” or “detectable moiety” is a composition detectable by radiological, spectroscopic, photochemical, biochemical, immunochemical, chemical, or other physical means. For example, useful labels include radioisotopes such as 32P, fluorescent dyes, electron-dense reagents, enzymes (e.g., as commonly used in an ELISA), biotin, digoxigenin, or haptens and proteins that can be made detectable, e.g., by incorporating a radioactive component into a polypeptide or used to detect antibodies specifically reactive with the polypeptide. Typically a detectable label is a heterologous moiety attached to a probe or a molecule (e.g., a protein or nucleic acid) with defined binding characteristics (e.g., a polypeptide with a known binding specificity or a polynucleotide), so as to allow the presence of the probe/molecule (and therefore its binding target) to be readily detectable. The heterologous nature of the label ensures that it has an origin different from that of the probe or molecule that it labels, such that the probe/molecule attached with the detectable label does not constitute a naturally occurring composition (e.g., a naturally occurring polynucleotide or polypeptide sequence).

The term “recombinant” when used with reference, e.g., to a cell, or a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

As used herein, the term “cancer” encompasses various malignant neoplasms characterized by the proliferation of anaplastic cells that tend to invade surrounding tissue and metastasize to new body sites. Non-limiting examples of different types of cancer suitable for treatment using the compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

The term “immunotherapy” refers to any treatment that uses certain parts of a patient's immune system to fight diseases such as cancer. The patient's own immune system is stimulated (or suppressed), with administration of one or more agents for that purpose. In some cases, the immunotherapy is “targeted” or cancer cell-specific. In other cases, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (see, e.g., Kroemer et al. (2013), Annu. Rev. Immunol., 31:51-72). In addition, the term “immunogenic chemotherapy” further refers to any chemotherapy that results in priming the immune system such that it leads to enhanced immune activity towards cancer. Specific representative examples of consensus immunogenic chemotherapies include 5′-fluorouracil, anthracyclines, such as doxorubicin, and the platinum drug, oxaliplatin, among others.

The term “inhibiting” or “inhibition,” as used herein, refers to any detectable negative effect on a target biological process, such as RNA/protein expression of a target gene, the biological activity of a target protein, protein-protein specific binding or interaction, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process (e.g., a target cell proliferation rate), or any one of the downstream parameters mentioned above, when compared to a control. “Inhibition” further includes a 100% reduction, i.e., a complete elimination, prevention, or abolition of a target biological process or signal or disease/symptom. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal or disease/symptom. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or symptom/disease incidence.

As used in this application, an “increase” or a “decrease” refers to a detectable positive or negative change in quantity from a comparison control, e.g., an established standard control (such as an average rate of proliferation of a certain cell type), prior to a certain event (e.g., exposure to an agent regulating cellular proliferation). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” as well as terms indicating an action to cause such changes or differences, such as “increase,” “promote,” “enhance,” “decrease,” “inhibit,” and “suppress,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

As used herein, an “effective amount” or a “therapeutically effective amount” means the amount of an active agent that, when administered to a subject or patient for treating a disorder, is sufficient to prevent, reduce the frequency of, or alleviate the symptoms of the disorder. The effective amount will vary depending on a variety of the factors, such as a particular compound or bioactive agent used, the disease and its severity, the age, weight, and other factors of the subject to be treated. Amelioration of a symptom of a particular condition by administration of a pharmaceutical composition described herein refers to any lessening, whether permanent or temporary, that can be associated with the administration of the pharmaceutical composition. For example, the quantity of anti-FGFR4-Δ CAR-T cells administered is considered therapeutically effective for treating a condition involving undesired FGFR4-Δ expression when administration results in eliminated symptoms, delayed onset of symptoms, or reduced frequency or severity of symptoms including disease progression, for example, in the case of treating FGFR4-Δ-positive cancer, as indicated in tumor mass, metastasis, morbidity and mortality, etc. The exact amount “effective” for achieving a desired therapeutic effect will depend on the nature of the therapeutic agent, the manner of administration, and the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

A “subject,” or “subject in need of treatment,” as used herein, refers to an individual who seeks medical attention due to risk of, or actual sufferance from, a condition involving an undesirable or abnormal, excessive expression of FGFR4-Δ. The term subject can include both animals, especially mammals, and humans. Subjects or individuals in need of treatment include those that demonstrate symptoms caused by or related to FGFR4-Δ expression, e.g., undesirable or inappropriate cell proliferation, such as tumor and especially malignant tumor/cancer or are at risk of later developing these conditions and/or related symptoms.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agents effective for treating FGFR4-Δ-positive cancer by suppressing FGFR4-Δ-positive cell proliferation and/or survival in a subject is a composition that does not contain any other agents that may have any detectable positive or negative effect on the same target process (e.g., efficacy in treating FGFR4-Δ+ cancer or suppression of FGFR4-Δ+ cell proliferation/survival) or that may increase or decrease to any measurable extent of the disease severity or outcome among the receiving subjects.

A “promoter” is defined as an array of polynucleotide control sequences that direct transcription of another polynucleotide sequence. As used herein, a promoter includes necessary polynucleotide sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. In contrast, an “inducible” promoter is a promoter that rendered active by environmental or developmental regulation and under certain specific environmental or developmental conditions. The term “operably linked” refers to a functional linkage between a polynucleotide expression control sequence (such as a promoter, or array of transcription factor binding sites) and another polynucleotide sequence (such as a protein-coding sequence), wherein the expression control sequence directs transcription of the second polynucleotide sequence.

An “expression cassette” is a nucleic acid construct, generated recombinantly or synthetically, with a series of specified polynucleotide elements that permit transcription of a particular polynucleotide sequence in a host cell or in an in vitro transcription system (e.g., partially reconstituted cell lysate). An expression cassette may be the entirety or a part of a plasmid, viral genome, or other replicable nucleic acid construct such as episome. Typically, an expression cassette includes a polynucleotide sequence to be transcribed, operably linked to a promoter.

As used herein, the term “heterologous” is used to describe the relationship of two elements placed adjacent to each other in a construct, referring to these two elements such as two polynucleotide sequences (e.g., a promoter sequence and a polypeptide-encoding sequence) or two polypeptide sequences (e.g., a signal peptide sequence and another peptide sequence) being from two different natural origins, such that these two elements are not found in the same relative positions in nature. Thus, a “heterologous promoter” for a gene refers to a promoter that is not naturally operably linked to that gene. Similarly, a “heterologous polypeptide” or “heterologous polynucleotide” to one particular protein or its encoding sequence is one derived from an origin different from the protein's origin. The fusion of two heterologous polypeptide (or polynucleotide) sequences does not result in a longer polypeptide (or polynucleotide) sequence that can be found in nature as an intact protein (or naturally occurring nucleotide sequence) or a segment thereof.

The term “conjugated with” or “coupled to” describes any construction whereby the polypeptide, e.g., a polypeptide of this invention comprising an antigen-bind site capable of specifically binding to FGFR4-Δ, is covalently or non-covalently linked, attached, or joined at its N- or C-terminus or at any side reactive group to a moiety that may serve as a solid support, a detectable element, or a therapeutically active agent for the purpose of detecting, isolating, or imaging of target cells or tissue or for the purpose of treating a relevant medical condition.

The term “about” denotes a range of +/−10% of a pre-determined value. For example, “about 10” indicates a range of 90% to 110% of 10, i.e., 9 to 11.

### DETAILED DESCRIPTION OF THE INVENTION

### I. Introduction

The present invention relates to compositions and methods for treating diseases, disorders, or conditions associated with dysregulated expression of the FGFR4-Δ isoform. The invention provides a novel single-chain variable fragment (scFv) that specifically binds to FGFR4-Δ isoform. The invention also provides a fully human chimeric antigen receptor (CAR) that specifically targets FGFR4-Δ.

The FGFR4-Δ isoform was discovered for the first time by the present inventors. In contrast to the full-length isoform FGFR4-FL, which is expressed in both cancers and non-cancerous tissues, this FGFR4-Δ variant due to alternative splicing is observed overexpressed on solid tumors (such as HCC) where it appears to promote tumorigenesis, progression, and metastasis and predicts poor disease prognosis, indicating that it can be used as a specific target for development of CAR-T immunotherapy. The present inventors have demonstrated in this study that the anti-FGFR4-Δ CAR-T cells have a higher viability compared with anti-FGFR4 CAR-T and elicit a stronger cytotoxicity to FGFR4-Δ-positive cells, indicating anti-FGFR4-Δ scFv derived CAR-T as a safe and efficient solution for tumor immunotherapy.

### II. Recombinant Technology in General

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983). Polynucleotide sequences such as synthetic oligonucleotides can be verified using well-established methodologies, e.g., the chain termination method for sequencing double-stranded templates of Wallace et al., Gene 16:21-26 (1981).

### III. FGFR4-Δ Antibody Constructs

The present invention relates to the making and use of antibody constructs that specifically recognize and bind the FGFR4-Δ variant of the FGFR4 protein. In particular, an FGFR4-Δ antibody construct is derived from an anti-FGFR4-Δ monoclonal antibody, contains at least one set of six CDRs, three in each of the V_{H} and V_{L}, and therefore retains the binding specificity and/or affinity of the original monoclonal antibody for the FGFR4-Δ antigen. The CDRs can be found in Table 1 (IMGT numbering system) and Table 2 (Kabat numbering system). The FGFR4-Δ antibody constructs of this invention in some cases are characterized by the presence of at least one or both of the full variable region of the heavy chain and light chain, see SEQ ID NOs: 1-11 and 12-22, respectively. Optionally, the FGFR4-Δ antibody constructs of this invention are humanized.

A. scFv and CAR

One embodiment of the FGFR4-Δ antibody construct of the present invention is a single chain antibody or scFv, where at least one full set of six CDRs, i.e., CDR1-3 from each of the V_{H} and V_{L} of the original monoclonal antibody, is retained. The framework regions of each of the V_{H} and V_{L} are optionally modified, such as for humanization purposes. In some cases, at least one copy of each of the full length V_{H} and V_{L} is retained. In some cases, at least one copy of at least a portion of each of V_{H} and V_{L} (e.g., the portion containing all 3 CDRs) is included. They may be connected directly or optionally connected through a peptide linker, which has a length of 2-30 amino acids, usually no more than 20 or 25 amino acids. An exemplary scFv of the present invention is described herein with its amino acid sequence set forth in SEQ ID NO:23, which may be with or without a signaling peptide (e.g., SEQ ID NO:35) at the N-terminus. In some cases, the scFv comprises any one of the amino acid sequences of SEQ ID NOs: 23-33.

Another embodiment of the FGFR4-Δ antibody construct of the present invention is a chimeric antigen receptor or CAR, which includes at least an extracellular antigen-binding ectodomain, a transmembrane domain, and an intracellular endodomain. The extracellular ectodomain contains the antigen-binding site, characterized by the presence of at least one full set of six CDRs, i.e., CDR1-3 from each of the V_{H} and V_{L} of the original monoclonal antibody. For example, the ectodomain may comprise the anti-FGFR4-Δ scFv as described above and herein. On the other hand, the intracellular signaling domain includes at least the CD3 ζ chain of a T cell receptor (TCR) complex, optionally one and two additional costimulatory domains. In one example, the CAR construct of the present invention includes anti-FGFR4-Δ scFv, CD28 transmembrane domain, and CD3 T cell activating domain. One or more peptide linkers (e.g., SEQ ID NO:34) may be used between any two adjacent domains of the CAR construct. An exemplary CAR of the present invention is described herein contains within its amino acid sequence an scFv sequence (e.g., SEQ ID NO:23), CD28 transmembrane domain (e.g., SEQ ID NO:36), and CD3ζ T cell activating domain (e.g., SEQ ID NO:37). Optionally, a signaling peptide (e.g., SEQ ID NO:36) may be used at the N-terminus.

### B. Conjugates

In view of the prevalence of FGFR4-Δ expression in various conditions and diseases, especially in different types of cancers, and the desirable binding profile of the FGFR4-Δ antibody constructs of this invention, different forms of conjugates can be devised for use in the detection of FGFR4-Δ and in targeted delivery of therapeutic agents to FGFR4-Δ+ cells, thus providing new and effective means in the diagnosis and treatment of conditions and diseases involving inappropriate FGFR4-Δ expression.

More specifically, the FGFR4-Δ antibody construct of this invention as a polypeptide-based construct can be conjugated with a variety of additional moieties, polypeptide or non-polypeptide in nature, depending on the intended use of the conjugated FGFR4-Δ polypeptide antibody construct. Such a conjugate may involve a polypeptide of this invention, comprising an antigen-bind site capable of specifically binding to FGFR4-Δ, being linked, attached, or joined at its N- or C-terminus or at any side reactive group to a moiety to serve as a solid support, a detectable element, or a therapeutically active agent. The linkage may be provided by way of a covalent bond or a non-covalent bond, e.g., through the binding action between a known binding pair, such as biotin-streptavidin or SpyCatcher-SpyTag pair, with one binding partner affixed to the polypeptide of this invention and the other binding partner affixed to the moiety to be conjugated to the polypeptide of this invention. Once made, the conjugate may be used for the purpose of detecting, isolating, or imaging of FGFR4-Δ-expressing cells or tissues or for the purpose of treating a medical condition involving FGFR4-Δ expression. In this regard, a detectable moiety produces a signal permitting easy detection by radiological, spectroscopic, photochemical, biochemical, immunochemical, chemical, or other means. Therapeutic moieties suitable for use in this invention may include known effective agents for treating conditions or diseases involving FGFR4-Δ expression, such as anti-cancer therapeutic agents known in the art or described herein, e.g., for radiotherapy, chemotherapy, immunotherapy and the like.

### IV. Nucleic Acids and Host Cells

Upon completion of designing an FGFR4-Δ antibody construct of this invention (or its conjugate in the fusion polypeptide form), a nucleic acid comprising a polynucleotide sequence encoding the construct (or its conjugate) may be constructed. Such nucleic acid may be in the form of DNA or RNA. In some cases, the nucleic acid takes the form of an expression cassette in which the coding sequence is operably linked to a promoter, typically heterologous promoter, directing the transcription and/or expression of the FGFR4-Δ antibody construct. In some embodiments, the nucleic acid is a vector, especially an expression vector, encoding the FGFR4-Δ antibody construct, such as a plasmid or a viral vector.

### A. Expression Cassettes and Vectors

To obtain high level expression of a nucleic acid construct encoding a desired polypeptide, one typically subclones a polynucleotide sequence encoding the polypeptide into an expression cassette, e.g., an expression vector, that contains a strong promoter to direct transcription, a transcription/translation terminator, and a ribosome binding site for translational initiation. Suitable promoters are well known in the art and described, e.g., in Sambrook and Russell, supra, and Ausubel et al., supra. Eukaryotic and prokaryotic expression systems for bacterial, mammalian, yeast, insect, or plant cells are well known in the art and are also commercially available. Kits for such expression systems are commercially available. One exemplary eukaryotic expression vector is an adenoviral vector, an adeno-associated vector, or a retroviral vector.

### B. Host Cells and Recombinant Protein Expression

In the context of practicing the present invention, a broad variety of host cells, prokaryotic or eukaryotic, may be used for the expression and/or production of an FGFR4-Δ antibody construct of this invention. The choice of host cells will depend on the specific purpose of the expression, for example, whether it is for making an antibody construct (e.g., an anti-FGFR4-Δ scFv) or for producing T cells expressing an anti-FGFR4-Δ CAR construct to be used in CAR-T therapy.

Standard transfection methods can be used to produce bacterial, mammalian, yeast, insect, or plant cell lines that express large quantities of a recombinant polypeptide (e.g., an FGFR4-Δ antibody construct), which is then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing a polynucleotide sequence encoding a protein of interest into a host cell (see, e.g., Sambrook and Russell, supra). It is only necessary that the particular genetic engineering procedure used be capable of successfully introducing at least one coding sequence into the host cell capable of expressing the recombinant polypeptide.

When a recombinant polypeptide, e.g., an anti-FGFR4-Δ scFv of this invention, is expressed in host cells in satisfying quantity, its isolation/purification can follow the standard protein purification procedure including solubility fractionation, size differential filtration, and column chromatography, as well as various immunoassay-based methods.

For the purpose of producing recombinant immune effector cells expressing FGFR4-Δ, such as CAR-T cells of this invention, typically the desired type of immune effector cells, such as natural killer (NK) cells and T cells, including cytotoxic T lymphocytes (CTLs) and regulatory T cells, is first isolated from the peripheral mononuclear cells (PBMCs) taken from a patient being treated for a condition or disease characterized by the inappropriate expression of FGFR4-Δ. Alternatively, the immune effector cells may be obtained from another human subject, preferably with a genetic background similar to that of the intended recipient. The isolated cells are then transformed or transfected with an expression vector, e.g., a viral vector derived from an adenovirus, an adeno-associated virus (AAV), a lentivirus, or a retrovirus, encoding the anti-FGFR4-Δ CAR construct such that sufficient FGFR4-Δ expression level can be detected and verified on the surface of these cells. The manipulated immune effector cells, now steadily expressing the anti-FGFR4-Δ CAR construct on their cell surface, are then cultivated for expansion ex vivo to reach an adequate quantity before they are administered back to the patient. Typically, roughly in the range of about 5×10⁴ to about 5×10⁷ of such CAR T cells are re-infused into the patient during each application. For example, about 1×10⁵ to about 3×10⁷, about 3×10⁵ to about 3×10⁷, about 2×10⁵ to about 2×10⁷, about 5×10⁶ to about 1×10⁷ CAR T cells are administered in each infusion.

In addition, recent developments in the CAR T cell-based therapy field allow for the direct and in vivo establishment within a patient's body of T cells expressing the anti-FGFR4-Δ CAR construct of this invention for treating a disease involving FGFR4-Δ expression. In such scenarios, a nucleic acid construct (such as an expression vector, including a viral vector derived from a viral genome) encoding the anti-FGFR4-Δ CAR construct of this invention may be administered systemically or locally to the patient in an effective quantity, optionally in a formulation with nanoparticles (e.g., lipid nanoparticles), for proper delivery and target T cell uptake/expression. See, e.g., Smith et al., Nature Nanotechnology 12 (8):813-820, 2017; Agarwal et al., Oncoimmunology 8 (12):e1671761, 2019; and Ledford, Nature 625 (7994):225-226, 2023.

### V. Pharmaceutical Compositions and Administration

The present invention also provides pharmaceutical compositions or physiological compositions comprising an effective amount of an FGFR4-Δ antibody construct, e.g., an anti-FGFR4-Δ scFv or a conjugate thereof, that can target FGFR4-Δ-expressing cells and in both prophylactic and therapeutic applications to address conditions and diseases involving FGFR4-Δ expression, e.g., different types of cancers. Such pharmaceutical or physiological compositions also include one or more pharmaceutically or physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249:1527-1533 (1990).

The pharmaceutical compositions of the present invention can be administered by various routes, e.g., subcutaneously, transdermally, intramuscularly, intravenously, intraperitoneally, or intratumorally. The preferred routes of administering the pharmaceutical compositions are through injection, especially local delivery by injection (e.g., intratumoral injection) to a relevant organ or tissue in a patient suffering from a solid tumor involving excessive and undesirable cellular proliferation at daily doses of about 10-100,000 μg, about 100-10,000 μg, or about 1,000-5,000 μg of the recombinantly produced anti-FGFR4-Δ antibody construct or conjugate for a 70 kg adult human per day. The appropriate dose may be administered in a single daily dose or as divided doses presented at appropriate intervals, for example as two, three, four, or more subdoses per day.

For preparing pharmaceutical compositions containing an FGFR4-Δ antibody construct or conjugate thereof, one or more inert and pharmaceutically acceptable carriers are used. The pharmaceutical carrier(s) can be in any form, although liquid form is preferred for formulations intended for injection. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Liquid pharmaceutical compositions include, for example, solutions, suspensions, and emulsions suitable for injection. Sterile buffer solutions of the active component (e.g., a recombinantly anti-FGFR4-Δ antibody construct or conjugate) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component in the desired solvent system, and then passing the resulting solution through a membrane filter to sterilize it or, alternatively, by dissolving the sterile active component in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions of an FGFR4-Δ antibody construct or a conjugate or host cells (e.g., T cells) expressing the antibody construct (e.g., an anti-FGFR4-Δ CAR construct) thereof can be administered for prophylactic and/or therapeutic purposes. In therapeutic applications, compositions are administered to a patient already suffering from a condition that involves excessive and undesirable cellular proliferation in an amount sufficient to prevent, cure, reverse, or at least partially slow or arrest the symptoms of the condition and its complications, such as the onset, progression, duration, and severity of the disease. An amount adequate to accomplish this is defined as a “therapeutically effective dose.” Amounts effective for this use will depend on the severity of the disease or condition and the weight and general state of the patient, but generally, for example, from about 10-100,000 μg, about 100-10,000 μg, or about 1,000-5,000 μg of the FGFR4-Δ antibody construct or a conjugate thereof per day for a 70 kg patient, with dosages from 1-1500 μg to about 20-100 μg of the FGFR4-Δ antibody or conjugate per day per kg for a patient being more commonly used.

In prophylactic applications, pharmaceutical compositions containing an adequate amount of the anti-FGFR4-Δ antibody construct/conjugate or a host cell expressing such construct (e.g., CAR-T cells) are administered to a patient susceptible to or otherwise at heightened risk of developing a disease or condition caused or exacerbated by undesirable and excessive cellular proliferation, in an amount sufficient to delay or prevent the onset of the symptoms. Such an amount is defined to be a “prophylactically effective dose.” In this use, the precise amounts of the FGFR4-Δ antibody construct or host cells again depend on the patient's state of health and weight, but generally range, for example, from about 10-100,000 μg, about 100-10,000 μg, or about 1,000-5,000 μg of the FGFR4-Δ antibody construct or a conjugate thereof per day for a 70 kg patient, with dosages from 1-1500 μg to about 20-100 μg of the FGFR4-Δ antibody or conjugate per day per kg for a patient being more commonly used.

Single or multiple administrations of the compositions can be carried out with dose levels and pattern being selected by the treating physician. In any event, the pharmaceutical formulations should provide a quantity of the FGFR4-Δ antibody construct of this invention or its conjugate sufficient to effectively address at least one targeted symptom of a condition or disease involving FGFR4-Δ expression in the patient, either therapeutically or prophylactically.

### VI. Anti-Cancer Therapeutic Agents

One potential use of the FGFR4-Δ antibody construct of this invention or a conjugate thereof is the treatment of cancers where the relevant tissues or cells are characterized in FGFR4-Δ expression, which may include various types of blood cancers (such as leukemia or lymphoma) or solid tumors (such as HCC).

In such applications, one or more of these previously known effective anti-cancer therapeutic agents, including those named in this application, may be administered concurrently with the FGFR4-Δ antibody construct or its conjugate to subjects in need of treatment. Optionally, the agent(s) may be used in combination with the FGFR4-Δ antibody construct of the present invention (e.g., an anti-FGFR4-Δ scFv or its conjugate) to suppress cancer growth, inhibit cancer metastasis, and facilitate remission from the disease. In the case of combination therapy, all of the active agents may be administered concurrently each in an effective amount, either together in a single composition or separately in two or more different compositions.

For example, various chemotherapeutic agents are known to be effective for use to treat various cancers. As used herein, a “chemotherapeutic agent” encompasses any chemical compound exhibiting suppressive effect against cancer cells, thus useful in the treatment of cancer. Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods disclosed herein also include cytostatic and/or cytotoxic agents.

Exemplary anti-cancer therapeutic agents include alkylating agents such as altretamine, bendamustine, busulfan, carboquone, carmustine, chlorambucil, chlormethine, chlorozotocin, cyclophosphamide, dacarbazine, fotemustine, ifosfamide, lomustine, melphalan, melphalan flufenamide, mitobronitol, nimustine, nitrosoureas, pipobroman, ranimustine, semustine, streptozotocin, temozolomide, thiotepa, treosulfan, triaziquone, triethylenemelamine, trofosfamide, and uramustine; anthracyclines such as aclarubicin, daunorubicin, doxorubicin, epirubicin, idarubicin, mitoxantrone, pirarubicin, valrubicin, and zorubicin; cytoskeletal disruptors (taxanes) such as abraxane, cabazitaxel, docetaxel, larotaxel, paclitaxel, taxotere, and tesetaxel; epothilones such as ixabepilone; histone deacetylase inhibitors such as vorinostat, romidepsin, and inhibitors of topoisomerase I such as belotecan, camptothecin, exatecan, gimatecan, irinotecan, and topotecan; inhibitors of topoisomerase II such as etoposide, teniposide, and tafluposide; kinase inhibitors such as bortezomib, erlotinib, gefitinib, imatinib, vemurafenib, and vismodegib; nucleotide analogs and precursor analogs such as azacitidine, azathioprine, capecitabine, cytarabine, doxifluridine, fluorouracil, gemcitabine, hydroxyurea, mercaptopurine, methotrexate, and tioguanine (formerly thioguanine); peptide antibiotics such as actinomycin and bleomycin; platinum-based agents such as carboplatin, cisplatin, dicycloplatin, oxaliplatin, nedaplatin, and satraplatin; retinoids such as alitretinoin, bexarotene, and tretinoin; and vinca alkaloids and derivatives such as vinblastine, vincristine, vindesine, and vinorelbine.

In addition to targeting FGFR4-Δ, other immunotherapeutic approaches may also be used in combination for cancer treatment. In accordance with the strategy of active immunotherapy, for example, monoclonal antibodies and their conjugates can be used to target other cancer markers. They include adotrastuzumab (HER2), alemtuzumab (CD52), bevaclzumab (VEGF), brentuximab (CD30), capromab (PSMA), cetuximab (EGFR), elotuzumab (SLAMF7), ibritumomab (CD20), necitumumab (EGFR), obinutumab (CD20), ofatumumab (CD20), olaratumab (PDGFRA), panitumumab (EGFR), pertuzumab (HER2), ramucirumab (VEGFR2), rituximab (CD-20), trastuzumab (HER-2), inotuzumab-ozogamicin (CD22), gemtuzumab-ozogamicin (CD33), and bevacizumab-awwb (VEGF). Moreover, checkpoint inhibitors and cytokines can be used in accordance with the strategy of passive immunotherapy. Currently approved checkpoint inhibitors target molecules CTLA4, PD-1, and PD-L1, including ipilimumab (CTLA4), nivolumab, pembrolizumab, cemiplimab, spartalizumab (PD-1), atezolizumab, avelumab, and durvalumab (PD-L1). Cytokines for use in the treatment of cancer and associated conditions include granulocyte colony-stimulating factor (G-CSF), granulocyte macrophage colony-stimulating factor (GM-CSF), interleukin-2 (IL-2), and interleukin-11 (IL-11).

### VII. Kits and Compositions

The invention provides compositions and kits comprising the anti-FGFR4-Δ antibody construct of this invention for practicing the methods described herein for detecting/assessing FGFR4-Δ expression in cells or tissues, for isolating FGFR4-Δ protein, and for treating diseases or conditions relating to FGFR4-Δ expression, including various types of malignancy.

Kits for carrying out assays for detecting/assessing FGFR4-Δ expression or for isolating FGFR4-Δ protein typically include one container containing an FGFR4-Δ antibody construct (e.g., an anti-FGFR4-Δ scFv), optionally conjugated to a solid support or a detectable moiety, such that the specific binding relationship between the FGFR4-Δ antibody construct and FGFR4-Δ would permit ready isolation and/or detection of FGFR4-Δ protein, both qualitatively and quantitatively, especially in an environment where another variant of FGFR4 (e.g., FGFR4-FL) might be present. In embodiments where the FGFR4-Δ antibody construct is not conjugated to any moiety to facilitate isolation or detection, the kits often contain a second container containing a detection agent that is able to specifically detect the presence of the FGFR4-Δ antibody construct.

Typically, the kits for detection or isolation of FGFR4-Δ also include one or more appropriate controls, indicative of the presence of FGFR4-Δ protein in a certain amount or below a detection threshold for the protein. In some cases such control(s) may be provided in the form of one or more physical samples, whereas in other cases the control(s) may be provided as a set value or values. In addition, the kits of this invention may provide instruction manuals to guide users in first processing test samples and then assessing the expression profile of FGFR4-Δ protein or isolating FGFR4-Δ protein from the samples.

Kits for practicing the methods described herein to treat a disease or condition caused or exacerbated by FGFR4-Δ expression via administering an FGFR4-Δ antibody construct or a conjugate thereof to a subject in need thereof. Both therapeutic use and prophylactic use are contemplated, i.e., a subject with or without a disease diagnosis may be treated, for example, once the presence and/or risk of the disease in the subject has been assessed according to the methods described herein and the subject is deemed to likely benefit from the treatment.

Kits for therapeutic use of an FGFR4-Δ antibody construct or a conjugate thereof typically include one container containing a composition comprising the FGFR4-Δ antibody construct, its conjugate, or a host cell expressing an FGFR4-Δ antibody construct (e.g., anti-FGFR4-Δ CAR T cell). Typically, such composition is formulated for delivering the FGFR4-Δ antibody construct, its conjugate, or a host cell expressing an FGFR4-Δ antibody construct, e.g., by injection such as via subcutaneous, intravenous, intramuscular, intraperitoneal, or intratumoral means. In some cases, the FGFR4-Δ antibody construct is conjugated with a therapeutic agent effective for treating conditions and diseases involving FGFR4-Δ expression, including cancer, such that a targeted delivery of the therapeutic agent can be achieved. In some cases, especially when host cells expressing an FGFR4-Δ antibody construct are included in the kits, at least one, possibly two or more additional containers may be included in the kits, each container containing at least one therapeutic agent known for its effectiveness in treating a condition or disease characterized by FGFR4-Δ expression. For example, when treatment of malignancy is sought, one or more anti-cancer therapeutic agents may be included in the kits. In particular, any one or more of the anti-cancer therapeutic agents known/used in the medical field or described herein may be included, especially chemotherapeutic drugs, e.g., drugs capable of killing or suppressing cells that are actively undergoing proliferation, and immunotherapeutic agents, e.g., checkpoint inhibitors and cytokines.

Further, the kits of this invention may provide instruction manuals to guide users in the proper administration of the composition comprising the FGFR4-Δ antibody construct, its conjugate (e.g., anti-FGFR4-Δ conjugated with a therapeutic agent), or a host cell expressing an FGFR4-Δ antibody construct (e.g., anti-FGFR4-Δ CAR T cell) to a subject deemed in need of such treatment by a physician (e.g., a person suffering from a condition involving FGFR4-Δ expression, including certain types of malignancy), the schedule (e.g., dose and frequency of administration) and route of administration, and the like.

As the alternative splicing variant FGFR4-Δ was previously unknown until being characterized by the present inventors for the first time, this invention in addition provides a composition comprising (a) the FGFR4-Δ protein having the amino acid sequence set forth in SEQ ID NO:41 (which may be optionally linked to a heterologous tag, such as one for ease of purification or detection, e.g., a poly-His tag); (b) a nucleic acid comprising a polynucleotide sequence encoding the FGFR4-Δ protein (such as an expression cassette comprising a polynucleotide sequence encoding the FGFR4-Δ protein operably linked to a promoter (for example, a heterologous promoter), or a vector comprising the expression cassette; or (c) a host cell (especially a recombinantly produced host cell) comprising the expression cassette or vector described in (b), thus containing the FGFR4-Δ protein and/or its coding sequence.

### Examples

The following examples are provided by way of illustration only and not by way of limitation. Those of skill in the art will readily recognize a variety of non-critical parameters that could be changed or modified to yield essentially similar results.

### INTRODUCTION

Hepatocellular carcinoma (HCC) is the most common form of primary liver cancer and remains a leading cause of cancer-related deaths worldwide (1). Despite advancements in early detection and treatment strategies, many HCC cases are diagnosed at advanced stages, where treatment options are limited (1). Current systemic therapies, such as kinase inhibitors and immune checkpoint inhibitors, have shown only modest clinical benefits (2-4), highlighting the unmet clinical need for selective and effective treatment strategies. One of the major challenges in developing targeted therapies for HCC is the lack of tumor-specific antigens that differentiate malignant cells from normal liver tissue. This makes it difficult to achieve tumor-specific targeting while minimizing off-target effects.

Alternative splicing (AS) is a crucial mechanism that increases the diversity of proteins encoded by the genome by selectively including or excluding specific exons during mRNA processing. Tumor cells show a significantly higher frequency of aberrant splicing events, producing cancer-associated isoforms that are either absent or minimally expressed in normal tissues (5). These tumor-specific splicing variants represent an underexplored source of novel antigens that could serve as selective targets for antibody-based therapies and diagnostics. Our team previously deployed hybrid sequencing, which integrates long-read RNA sequencing to capture full-length transcripts and short-read RNA sequencing for quantification, to map HCC-associated splicing landscape and validated a number of tumor-specific splicing variants (6). By expanding this approach to patient-derived HCC organoids, we identified a previously unannotated splice variant of fibroblast growth factor receptor 4 (FGFR4), which we designated as FGFR4-4.

FGFR4 is a transmembrane receptor essential for liver function, particularly in bile acid metabolism and cellular proliferation through the FGF19-FGFR4-β-Klotho signaling axis. Dysregulation of this pathway promotes tumor growth, invasion, and metastasis in HCC (7). Upon FGF19 binding, FGFR4 undergoes dimerization, activating oncogenic signaling cascades, including Ras/Raf-MEK-MAPK, PI3K/AKT, and STATs, while also enhancing metastatic potential via GSK3β-β-catenin signaling (7). Although pan-FGFR inhibitors such as Fisogatinib and Erdafitinib have been developed to block FGFR4-driven tumor progression, their clinical efficacy is limited due to off-target effects on normal FGFR4 functions, leading to metabolic toxicity and bile acid dysregulation (8). The identification of the tumor-specific FGFR4 variant provides a new avenue for selectively targeting FGFR4-mediated oncogenic pathways while minimizing toxicity.

Notably, FGFR4-Δ has not been documented in public repositories. In this study, we assessed its expression, clinical relevance and biological functions in HCC. We also developed FGFR4-Δ specific monoclonal antibodies and CAR-T therapies to selectively target FGFR4-Δ-positive HCC cells. Our findings identify FGFR4-Δ as a tumor-specific antigen with strong oncogenic potential and provide a foundation for the development of FGFR4-Δ-targeted immunotherapies for HCC.

### Methods and Materials

### Cell Lines and Culture Conditions

HCC tumor organoids were cultured in Basal medium, which is AIM-V medium supplemented with 16% FBS, 0.35 U/ml Pen/Strep, 1% L-Glutamine, 1% MEM non-essential amino acids (NEAA), and 0.2% insulin-transferrin-selenium (ITS). HCC cell lines expressing FGFR4-Δ (HCC-FGFR4A and HCC-FGFR4Δ-GL, with GL indicating stable expression of GFP and luciferase) were maintained in AIM V medium from Gibco, also supplemented with 10% FBS, 2 mM L-glutamine, and 100 U/mL of Penicillin-Streptomycin. Primary human T cells were cultured in GT-T551 H3 medium from Takara Bio-station, supplemented with 10% FBS and 100 U/mL of Penicillin-Streptomycin. Human recombinant interleukin-2 (hIL-2) was sourced from PeproTech at a concentration of 300 IU/mL and was added to the T cell cultures to promote expansion. HEK-293T cells were employed for lentivirus production and were cultured in Dulbecco's Modified Eagle Medium (DMEM) from Gibco (Life Technologies), supplemented with 10% fetal bovine serum (FBS), 2 mM L-glutamine, 50 μM β-mercaptoethanol, and 100 U/mL of Penicillin-Streptomycin.

### Total RNA Extraction

Total RNA from liver tissues were extracted using Qiagen RNeasy Plus Mini Kit (Catalog Number: 74034). Tissues were minced on dry ice with scalpels precooled in −80 °C freezer. Six hundred microliters of Buffer RLT Plus was added to the minced tissues. After vertexing and centrifugation, the supernatant was transferred to gDNA eliminator spin columns to eliminate genomic DNA. Six hundred microliters of 70% Ethanol was added to the flow-through and mixed by pipetting up and down. The mixture was then transferred to RNeasy spin columns and centrifuged at 8,000 g for 15 seconds at room temperature. The columns were washed once with 700 μL Buffer RW1, and then twice with 700 μL Buffer RPE. The columns were spun for 2 min at 15,000 g in order to thoroughly dry the resin. At last, RNA was eluted from the columns with 30 μL Nuclease-free water. The purified RNA samples were placed at −80 °C for long term storage. The concentration and purity of the extracted RNA were determined by measuring optical density (OD) at 260 nm and ration of OD 260/280, respectively, using the ND-2000 UV-VIS Spectrometer.

### Reverse Transcription

SuperScript™ III First-Strand Synthesis System kit (Invitrogen, Catalog Number: 18080051) was used to conduct reverse transcription. Five hundred nanograms of RNA sample was mixed with 1 μL dNTP mix (10 mM) and 1 μL Random hexamers (50 ng/μL). The mixture was incubated at 65 °C for 5 min, and then placed on ice for at least 1 min. After mixed with 2 μL 10×RT buffer, 4 μL 25 mM MgCl2, 2 μL 0.1M DTT, 1 μL RNaseOUT (40 U/μL) and 1 μL SuperScript® III RT (200 U/μL), the mixture was incubated for 10 min at 25 °C, followed by 50 min at 50 °C. The reaction was terminated at 85 °C for 5 min and then chilled on ice. At last, 1 μL RNase H was added into each tube and incubated for 20 min to eliminate residual RNA. After diluted 10 times with Nuclease-free water, cDNA samples were placed at −20 °C for long term storage.

TaqMan-Based Quantitative Real-Time PCR (qPCR)

TaqMan™ Universal PCR Master Mix (Applied Biosystems, Catalog Number: 4304437) was used. Forward primer (SEQ ID NO:38) and reverse primer (SEQ ID NO:39) of FGFR4-Δ will be dissolved in nuclease-free waster at concentration of 10 μM. Taqman probe of FGFR4-Δ (SEQ ID NO: 40) will be dissolved in nuclease-free waster at concentration of 25 μM. Expression for FGFR4-Δ and endogenous control 18S were determined in separate wells. To determine expression of FGFR4-Δ, each reaction was consisted of 2 μL diluted cDNA, 5 μL TaqMan™ Universal PCR Master Mix, 1 μL forward primer, 1 μL reverse primer, 0.25 μL Taqman probe and 0.75 μL Nuclease-free waster. To determine expression of endogenous control 18S, each reaction was consisted of 2 μL diluted cDNA, 5 μL TaqMan™ Universal PCR Master Mix, 0.5 μL 20× TaqMan Gene Expression assay and 2.5 μL Nuclease-free waster. All assays were conducted in triplicate wells of 384-well PCR plates (Thermo Scientific, Catalog Number: AB1384). QuantStudio™ 7 Flex Real-Time PCR System (Applied Biosystems) was used to perform qPCR.

### Enzyme-Linked Immunosorbent Assay (ELISA) for Antibody Binding Assessment

The binding ability of the anti-FGFR4-Δ antibodies was evaluated using ELISA. High-binding 96-well ELISA plates were coated with either FGFR4-Δ-6His or FGFR4-full-length-6His protein at a concentration of 0.25 μg/mL and incubated overnight at 4 °C. After incubation, the wells were washed three times with PBS containing 0.05% Tween-20 (PBST), followed by blocking using 200 μL of 5% bovine serum albumin (BSA) in PBS for one hour at room temperature. After that, the antibodies were prepared in a two-fold serial dilution starting from a concentration of 1 μg/mL. A total of 100 μL of each antibody dilution was added to the wells and incubated for one hour at room temperature. After washing three times with PBST, a goat anti-mouse IgG Fc-specific secondary antibody conjugated to horseradish peroxidase (HRP) was added to each well and incubated for 45 minutes at room temperature. The wells were then washed five times with PBST before adding 100 μL of tetramethylbenzidine (TMB) substrate solution. After incubation in the dark for 10 minutes, reactions were terminated by adding 50 μL of 2M sulfuric acid. Absorbance was measured at 450 nm using a microplate reader.

### Flow Cytometry Analysis

Flow cytometry analysis was performed to assess the binding specificity of the generated antibodies to FGFR4-Δ. HCC cells overexpressing FGFR4-Δ were harvested, washed, and resuspended in phosphate-buffered saline (PBS) containing 1% bovine serum albumin (BSA). The cells were then incubated with hybridoma supernatants or purified antibodies at 4 °C for 30 minutes. After incubation, the cells were washed to remove any unbound antibodies and subsequently stained with a fluorochrome-conjugated secondary antibody for 30 minutes at 4 °C in the dark. Following a final wash, the cells were analyzed using a flow cytometer.

Generation of FGFR4-Δ scFv Construct

To construct the anti-FGFR4-Δ CAR, we first synthesized the anti-FGFR4-Δ scFv (selected from SEQ ID NO:23 to SEQ ID NO:33), including the heavy chain sequence (selected from SEQ ID NO: 1 to SEQ ID NO:11) and light chain sequence (selected from SEQ ID NO:12 to SEQ ID NO:22) of anti-human FGFR4-Δ antibodies with a signal peptide (SEQ ID NO:35) and a linker (SEQ ID NO:34). To transduce human T lymphocytes to express anti-FGFR4-Δ CAR, the DNA sequence of anti-FGFR4-Δ scFv was inserted upstream of CD28 transmembrane domain sequence (SEQ ID NO:36), CD3ζT cell activating domain sequence (SEQ ID NO:37), 2A sequence and Green Fluorescent Protein (GFP) sequence in a lentivirus vector (pWPXLd-antiCD44E-CD28-CD3ζ-2A-GFP). The CAR fragment was inserted into the pWPXLd-eGFP vector by T4 DNA ligase. The CAR vectors were transformed into Stb13 competent cells, and the positive plasmid was selected and harvested for lentiviral expression of CARs.

### Lentivirus Production

Lentiviral particles were produced in HEK-293T cells using polyethyleneimine (PEI) for transfection (Sigma-Aldrich). HEK-293T cells were seeded in 10 cm dishes and transfected with a mixture containing the lentiviral plasmids along with two packaging plasmids, psPAX2 and pMD.2G, in a ratio of 3:4:1. A total of 24 μg of plasmid DNA was used per dish, combined with 36 μg of PEI. The transfection mixture was incubated at room temperature for 15 minutes before being added to the cells. The culture supernatants containing lentiviral particles were collected 24, 48, and 72 hours post-transfection, filtered through a 0.45-μm filter, and stored at −80 °C until needed.

### Isolation, Transduction, and Expansion of Primary Human T Cells

Peripheral blood mononuclear cells (PBMCs) were isolated from human blood samples using density gradient centrifugation with Lymphoprep (Stem Cell Technologies). Primary human T cells were enriched from PBMCs by negative selection using the Pan T Cell Isolation Kit (Miltenyi Biotec). Freshly isolated T cells were activated using T Cell TransAct beads (Miltenyi Biotec) at a cell-to-bead ratio of 1:2. Twenty-four hours after activation, T cells were transduced with lentiviral supernatants containing either control-CAR, FGFR4-Δ-CAR, or an empty vector control. Following transduction, T cells were cultured in GT-T551 H3 medium supplemented with hIL-2 (300 IU/mL) and expanded for seven to ten days before functional assays.

### In Vitro Cytotoxicity Assay

The cytotoxicity of FGFR4-Δ-CAR T cells was assessed using a luciferase-based cytotoxicity assay. HCC cells expressing empty vector, FGFR4-FL or FGFR4-Δ were firstly transduced with GFP-2A-luciferase construct and seeded in U-bottom 96-well plates, followed by co-cultured with FGFR4-Δ-CAR T cells or mock-transduced T cells at specified effector-to-target (E:T) ratios. After 24 hours of co-culture, the d-Luciferin substrate (Cayman Chemical) was added to the wells, and luminescence was measured with a microplate reader to evaluate target cell viability. The percentage of viable target cells was calculated using the formula: (experimental signal/maximal signal)×100. The percentage of tumor cell killing was then determined as 100 minus the viability percentage. All experiments were conducted in triplicate, and data were normalized to control wells.

### Subcutaneous Xenograft Models

To investigate the cytotoxicity of FGFR4-Δ-CAR T cells in vivo, 1×10⁶ HepG2 cells expressing either empty vector or FGFR4-Δ were suspended in 100 μl of Matrigel and subcutaneous injected into the right flank of male NSG mice (4-8 weeks old). After 3 weeks, each group of xenografts were randomly divided into two groups and subjected to the treatment of FGFR4-Δ CAR T cells or mock CAR-T cells through peritumoral injection. Tumor size was measured every 3 days for 24 days. Tumor volumes were calculated by the equation Volume=0.5×Length×Width². Animals were sacrificed at day 24 post CAR T treatment and tumor xenografts were harvested for further analysis.

### Statistics

Data are presented as the mean±SD. The results were analyzed via unpaired Student's t-test (two-tailed) and two-way ANOVA. Statistical significance was defined by a P-value of less than 0.05. All statistical analyses were performed using the Prism software version 6.0 (GraphPad).

### Results

### FGFR4-Δ was Identified in Primary HCC Tumor

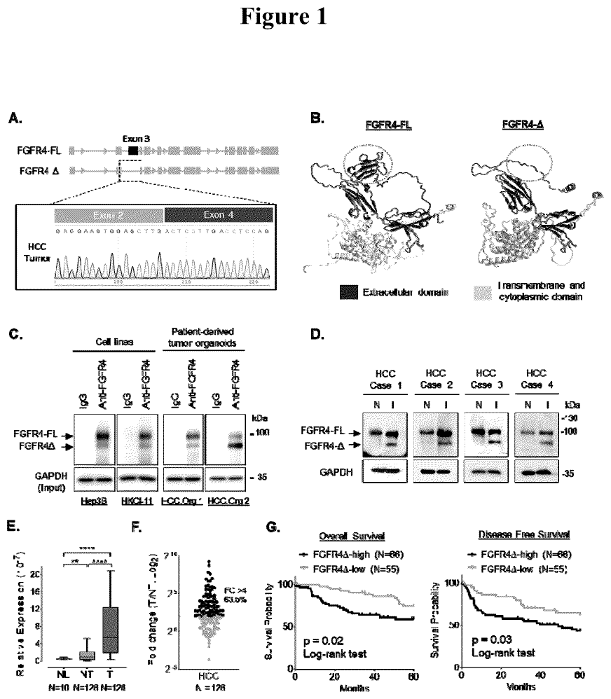

Results from our hybrid-sequencing revealed the tumor-specific expression of a novel splice variant of FGFR4, namely FGFR4-Δ. This variant exhibits a skipped exon 3 compared to the canonical FGFR4-full length (FL) variant (FIG. 1A) and predicted to be around 75 kDa. AlphaFold predictions indicated that FGFR4-Δ presented a distinct extracellular domain compared to FGFR4-FL (FIG. 1B), indicating the feasibility of specific targeting of FGFR4-Δ. Presence of FGFR4-Δ in HCC was corroborated by Sanger sequencing (FIG. 1A). To verify the protein abundance of this variant, we conducted pull-down assays with an FGFR4 antibody that targets the conserved C-terminus of the FGFR4 protein in HCC cell lines and patient-derived HCC organoids. The result revealed that FGFR4-FL and FGFR4-Δ variants are two dominant FGFR4 isoforms in HCC (FIG. 1C). This observation was further confirmed in 21 pairs of primary HCC tumors and adjacent non-tumoral liver tissues, where FGFR4-FL and FGFR4-Δ were both abundantly expressed in HCC tumor at a ratio ranging from 5:1 to 1:1 (FIG. 1D). Notably, FGFR4-Δ isoform was uniquely expressed in HCC tumors (FIG. 1D), whereas the canonical FGFR4-FL isoform, despite being overexpressed in tumors, was also present in non-tumoral liver tissues (FIG. 1D).

To extend our observations to a larger cohort, we designed juncture-specific TaqMan assays to measure the mRNA expression of FGFR4-Δ. The results showed that FGFR4-Δ were significantly upregulated in HCC tumors (FIG. 1E). Of note, 63.5% of HCC cases exhibited more than a four-fold increase in FGFR4-Δ expression in tumor tissues compared to adjacent non-tumor liver tissues (FIG. 1F), indicating its prevalence in HCC. To assess the clinical significance of FGFR4-Δ, HCC cases were classified into FGFR4-Δ-high (fold change≥4) and FGFR4-Δ-low (fold change<4) groups based on the FGFR4-Δ expression ratio in tumor tissues relative to adjacent non-tumor tissues. Our results revealed that high FGFR4-Δ significantly correlated with poorer overall survival (P=0.0195) and disease-free survival (P=0.0286) in HCC (FIG. 1G).

### FGFR4-Δ Promoted Viability, Colony Formation, and Migration in HCC

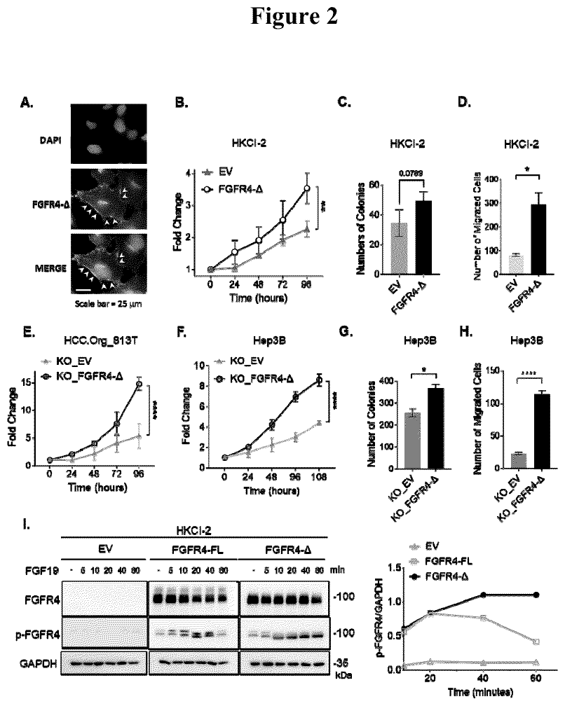

To investigate the biological function of FGFR4-Δ, we overexpressed this isoform in the HCC cell line HKCI-2, which exhibited low levels of FGFR4 isoforms. Immunofluorescence staining confirmed that FGFR4-Δ localizes to the cell membrane (FIG. 2A). Expression of FGFR4-Δ significantly enhanced cell proliferation, colony formation, and migration in HKCI-2 cells (FIGS. 2B-D).

To eliminate the influence of endogenous FGFR4 expression, we knocked out FGFR4 in HCC cell line Hep3B and patient-derived HCC organoid 813T (HCC.Org_813T), followed by FGFR4-Δ overexpression. Consistently, FGFR4-Δ exhibited a pronounced oncogenic effect, as evidenced by the enhanced proliferation, colony formation and migration compared to the empty vector control (FIGS. 2E-H). Moreover, upon FGF19 treatment, FGFR4-Δ induced an enhanced and more sustained phosphorylation level of FGFR4 protein compared with the canonical counterpart FGFR4-FL (FIG. 21). These findings demonstrated that FGFR4-Δ is specifically expressed in HCC tumor cells and actively contributes to HCC progression.

### Development and Characterization of the FGFR4-Δ Specific Antibodies

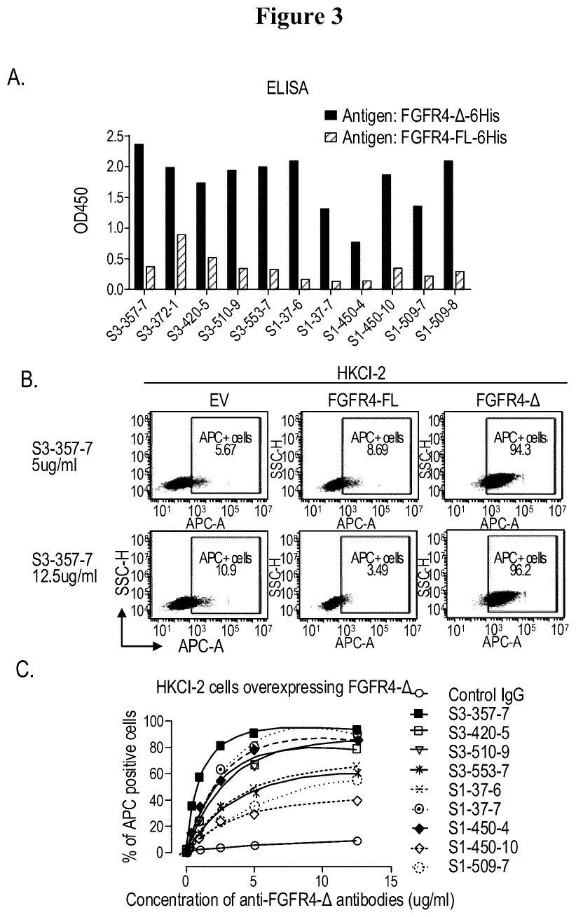

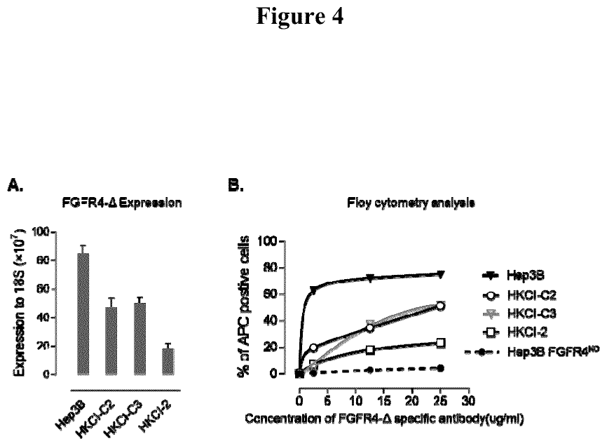

To selectively target FGFR4-Δ expressing cells, we first screened for FGFR4-Δ specific antibodies. Recombinant FGFR4-Δ and FGFR4-FL extracellular regions were expressed in CHO cells to generate purified antigens for immunization and antibody screening. Mice were immunized with the recombinant FGFR4-Δ protein, and hybridoma technology was used to generate monoclonal antibodies (mAbs). Using ELISA assays, we identified 11 anti-FGFR4-Δ antibodies exhibiting strong binding affinity to FGFR4-Δ, but minimal cross-reactivity with FGFR4-FL (FIG. 3A). Flow cytometry assays also confirmed that the selected antibodies specifically recognize the human FGFR4-Δ protein on the cell surface of HCC cells (FIGS. 3B and 3C). To evaluate the ability of these antibodies to detect endogenous FGFR4-Δ, we performed flow cytometry on HCC cell lines with varying levels of FGFR4-Δ expression (FIG. 4A), using antibody S3-357-7 as a representative antibody. The result demonstrated that antibody S3-357-7 bound to HCC cell lines in a manner consistent with their FGFR4-Δ mRNA expression levels (FIG. 4B). Importantly, no binding was observed in FGFR4-knockout (KO) Hep3B cells (FIG. 4B), which further confirms the specificity of antibody S3-357-7 for FGFR4-Δ. The results indicate that the developed antibodies exhibit both high specificity and affinity for the human FGFR4-Δ protein.

### Construction of Human Anti-FGFR4-Δ CAR-T Cells

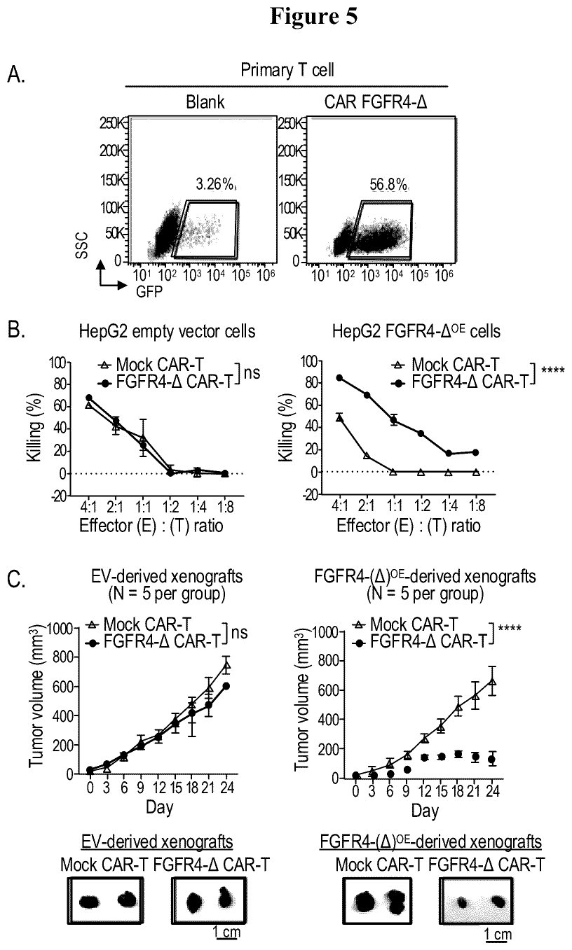

Immunotherapy, particularly adoptive cell transfer (ACT)-based immunotherapy, has revolutionized cancer treatment by utilizing T cells engineered with chimeric antigen receptors (CARs) to target tumor-associated antigens (9). To engineer FGFR4-Δ specific CAR-T cells, we inserted the single-chain variable fragment (scFv) sequence of anti-FGFR4-Δ antibody S3-357-7 antibody into a second-generation CAR vector. This vector incorporates the 4-1BB (CD137) costimulatory domain and the CD3ζ signaling domain to enhance T cell activation and persistence. The resulting construct, pWPXLd-scFv (S3-357-7)-CD28-CD3z-EGFP, was used to prepare lentiviral particles, which were subsequently used to transduce primary human T cells. Successful transduction was verified by flow cytometric analysis, which detected the expression of GFP co-expressed with the CAR molecule (FIG. 5A). So far, we have successfully generated FGFR4-Δ specific CAR-T cells with a transduction efficiency of approximately 56% (FIG. 5A).

### Cytotoxicity of FGFR4-Δ Specific CAR-T Therapy

To evaluate the cytotoxicity of FGFR4-Δ CAR in vitro, we co-cultured anti-FGFR4-Δ CAR-T or control CAR-T cells with GFP-2A-luciferase labelled FGFR4-Δ overexpressing-HepG2 cells at an effector-to-target (E/T) ratio ranging from 2:1 to 1:8 for 24 hours. Cell viability was assessed by measuring luciferase signal intensity. Compared to control CAR-T cells, anti-FGFR4-Δ CAR-T cells exhibited significantly enhanced cytolytic activity across a broad range of E/T ratios (4:1 to 1:8, FIG. 5B), indicating a cell-load dependent cytotoxic effect against FGFR4-Δ positive cell.

To validated the efficacy of FGFR4-Δ CAR-T cells in vivo, we established subcutaneous xenografts using either vector control or FGFR4-Δ-overexpressing-HepG2 cells and subjected them to CAR-T therapy. Following CAR-T injection, anti-FGFR4-Δ CAR-T cells demonstrated a marked and specific tumor-suppressive effect, as evidenced by a significant reduction in tumor volume in FGFR4-Δ-overexpressing xenografts compared to control CAR-T treatment (FIG. 5C). In contrast, no significant tumor suppression was observed in vector control xenografts (FIG. 5C). Taken together, these findings demonstrate the potent and target-specific cytotoxicity of FGFR4-Δ CAR-T therapy both in vitro and in vivo.

### REFERENCE

1. Mak L Y, Liu K, Chirapongsathorn S, Yew K C, Tamaki N, Rajaram R B, et al. Liver diseases and hepatocellular carcinoma in the Asia-Pacific region: burden, trends, challenges and future directions. Nat Rev Gastroenterol Hepatol. 2024 December; 21(12):834-51.
2. Llovet J M, Ricci S, Mazzaferro V, Hilgard P, Gane E, Blanc J F, et al. Sorafenib in advanced hepatocellular carcinoma. N Engl J Med. 2008 July; 359(4):378-90.
3. Kudo M, Finn R S, Qin S, Han K H, Ikeda K, Piscaglia F, et al. Lenvatinib versus sorafenib in first-line treatment of patients with unresectable hepatocellular carcinoma: a randomised phase 3 non-inferiority trial. The Lancet. 2018 March; 391(10126):1163-73.
4. Sangro B, Sarobe P, Hervas-Stubbs S, Melero I. Advances in immunotherapy for hepatocellular carcinoma. Nat Rev Gastroenterol Hepatol. 2021 August; 18(8):525-43.
5. Cheng R, Xiao L, Zhou W, Jin X, Xu Z, Xu C, et al. A pan-cancer analysis of alternative splicing of splicing factors in 6904 patients. Oncogene. 2021 September; 40(35):5441-50.
6. Chen H, Gao F, He M, Ding X F, Wong A M, Sze S C, et al. Long-read RNA sequencing identifies alternative splice variants in hepatocellular carcinoma and tumor-specific isoforms. Hepatology. 2019 September; 70(3):1011-25.
7. Raja A, Park I, Haq F, Ahn S M. FGF19-FGFR4 Signaling in Hepatocellular Carcinoma. Cells. 2019 Jun. 4; 8(6):536.
8. Kommalapati A, Tella S H, Borad M, Javle M, Mahipal A. FGFR Inhibitors in Oncology: Insight on the Management of Toxicities in Clinical Practice. Cancers. 2021 Jun. 13; 13(12):2968.
9. Albelda S M. CAR T cell therapy for patients with solid tumours: key lessons to learn and unlearn. Nat Rev Clin Oncol. 2024 January; 21(1):47-66.

All patents, patent applications, and other publications, including GenBank Accession Numbers or similar sequence identification numbers, cited in this application are incorporated by reference in the entirety of their contents for all purposes.

### SEQUENCE LISTING

Provided Below are the VH and VL Domain Sequences of FGFR4-Δ Monoclonal Antibodies. The CDR Sequences, as Determined by IMGT, are Shown in Bold.
SEQ ID NO:1 is the amino acid sequence of the V_{H} of the S3-357-7 anti-FGFR4-Δ mAb

EVKLQESGAELVKPGVSVKISCKTSGFTFSDYMDWVKQSHGKSLEWIGWI
NPNSGGTNYNQKFKGKATLTADKSSSTAYMQLSSLTSEDSAVYYCARFGY
DNSGAFFDYWGRGTLVTVSA

SEQ ID NO:2 is the amino acid sequence of the V_{H} of the S3-372-1 anti-FGFR4-Δ mAb

EVKLQQPGAELVRPGASVKISCKASGFTFSDYMHWVKQTHGKSLEWIGRI
IGNAEGGYYNQKFKGKATLTADKSSSTAYMQLSSLTSEDSAVYYCARDYF
YGSTFYFDYWGQGTLVTVSA

SEQ ID NO:3 is the amino acid sequence of the V_{H} of the S3-420-5 anti-FGFR4-Δ mAb

EVKLQNAGAELVKPGTSVKISCKRSGFTFSSYMDWVKQTHGKSLEWIGNI
TGNAGVAYYNQKFKGKATLTADKSSSTAYMQLSSLTSEDSAVYYCARVRD
TSGYNYFFDYGRGTLVTVSAA

SEQ ID NO:4 is the amino acid sequence of the V_{H} of the S3-510-9 anti-FGFR4-Δ mAb

EAKLEEAGAELVKPGATVRVTCKGSGFTFSRYMDWVKQSHGKSLEWIGVI
TGNAGWSYYNQKFKGKATLTADKSSSTAYMQLSSLTSEDSAVYYCARDYY
GNSYYFDYWGQGTLVTVSA

SEQ ID NO:5 is the amino acid sequence of the V_{H} of the S3-553-7 anti-FGFR4-Δ mAb

EVKLQKAGAELVKPGASVKISYRNSGFTFSRYMDWVKQSHGKSLEWIANI
KGNAGVGYYNQKFKGKATLTADKSSSTAYMQLSSLTSEDSAVYYCARVRD
TSGYNYFFDYWGQGTLVTVSA

SEQ ID NO:6 is the amino acid sequence of the V_{H} of the S1-37-6 anti-FGFR4-Δ mAb

EVKLQQSGAELVKPGASVKISCKASGFTFSSYMDWVKQSHGKSLEWIGVI
TGNSGWGYYNQKFKGKATLTADKSSSTAYMQLSSLTSEDSAVYYCARDYY
GNSYHFDYWGRGTLVTVSA

SEQ ID NO:7 is the amino acid sequence of the V_{H} of the Clone S1-37-7 anti-FGFR4-Δ mAb

EVKLEESGGGLVQPGGSLKLSCVASGFSVNTHWMNWVRQSPTKGLEWVAE
IRLKTNNYATHYAETVKGRFTITRDNSQSTVYLQMNSLRTEDTGIYYCTS
KYNDYFDVWGSGTTVTVSS

SEQ ID NO:8 is the amino acid sequence of the V_{H} of the Clone S1-450-4 anti-FGFR4-Δ mAb

EVKLEESGGALVQPGGSMKLSCVASGYTVNSHWMSWVRQSPTKGLEWVAE
IKLKSNKYASHFAESVKGRFTITRDNSQSTVYLQMNSLRAEDTGIYYCTS
KFSDYFDVWGAGSTVTVAS

SEQ ID NO:9 is the amino acid sequence of the V_{H} of the S1-450-10 anti-FGFR4-Δ mAb

EVKLEESGGGLVQPGGSMKLSCVASGFTVNTHWMNWVRQSPEKGLEWVAE
IRLKSNNYATHYAESVKGRFTISRDNSQSTVYLQMNNLRTEDTGIYYCTS
KYNWYFDVWGAGTTVTVSS

SEQ ID NO:10 is the amino acid sequence of the V_{H} of the Clone S1-509-7 anti-FGFR4-Δ mAb

EVKLEESGGGLVQPGGSMKLTCTASGYSVNTHFLNWVRQSPTKGLEWVAE
IRLRSNDYATHFAESVKGRFTITRDGSQSTVYLQMNHLRTEDTGIYYCTS
RYSDYFDVWGAGSTVTVAS

SEQ ID NO:11 is the amino acid sequence of the V_{H} of the Clone S1-509-8 anti-FGFR4-Δ mAb

EVKLEDSGGGLVQPGGSMKLSCIASGFAVNTYWMNWVRQSPTKGLEWVPE
IRVKSNGYATHYAEAVKGRFTITRDGSQSTVFLQMNNLRTEDTGIYYCTT
KFSDYFDVWGVGTTVTVAS

SEQ ID NO:12 is the amino acid sequence of the V_{L} of the S3-357-7 anti-FGFR4-Δ mAb

DIQMTQSPSSLSASVGDRVTITCRASQNVGNTYLNWYQEKPGKAPKLLIY
KLSRLHSGVPSRFSGSGSGTDFTLTISRLEPEDFAVYYCQHSREVPWTFG
QGTKVEIK

SEQ ID NO:13 is the amino sequence of the V_{L} of the S3-372-1 anti-FGFR4-Δ mAb

DIQMTQTTSSLSAVSGDRVTITCRASQDVAGNTYLNWYQHKPGKAPKLLV
YKLSSRPSGVPSRFSGSGSGTDFTLTISRLEPEDFAVYYCQQWTYVPYTF
GSGTKLEIK

SEQ ID NO:14 is the amino acid sequence of the V_{L} of the S3-420-5 anti-FGFR4-Δ mAb

DIVMTQSPSSLTASVGDRVTITCRASQSVSGNTYLNWYQQKPGKTPKLLI
YQISRLTGGVPSRFSGSGSGTDFTLTISRLEPEDFAVYYCMRHMDYPFGA
GTKLDIK

SEQ ID NO:15 is the amino acid sequence of the V_{L} of the S3-510-9 anti-FGFR4-Δ mAb

DIVMTQSPSSLSASVGDRITVTCRASQDVSGNTYLNWYQQKPGRAPKLLI
YYTSRLPSGVPSRFSGSGSGTDFTLTISRLEPEDFAVYYCQQFSYVPYTF
GSGTKLEIK

SEQ ID NO:16 is the amino acid sequence of the V_{L} of the S3-553-7 anti-FGFR4-Δ mAb

DILMTQSPSSLSASVGDRVTITCRTSQSVSGNTYLNWYQQKPGKAPKFLI
YYTSRLTGGVPSRFSGSGSGTDFTLTISRLEPEDFAVYYCQHWSYVPYTF
GQGTKLEIK

SEQ ID NO: 17 is the amino acid sequence of the V_{L} of the S1-37-6 anti-FGFR4-Δ mAb

DIVLTQSPSSLSASVGERVTITCRASQEVSGDTYLNWYQHKPGKTPLLLI
YQISRLPSGVPSRFSGSGSGTDFTLTISRLDPEDFAVYYCQQWSYVPFTF
GSGTKLDIK

SEQ ID NO:18 is the amino acid sequence of the V_{L} of the Clone S1-37-7 anti-FGFR4-Δ mAb

DIVMTQAAPSISVTPGESVSISCRSSQDVSGHTYLNWYQQKPGKAPKLLI
YKLSRLPSGVPSRFSGSGSGTDFTLTISRMEPEDFAVYYCQQWSYMPFTF
GSGTKLEIK

SEQ ID NO:19 is the amino acid sequence of the V_{L} of the Clone S1-450-4 anti-FGFR4-Δ mAb

DIVMTQSPSSLSASVGDRVTITCRASQSVSGNTYLNWYQRKPGKAPKLLI
YYTSRLTGGVPSRFSGSGSGTDFTLTISRLEPEDFAVYYCQHWSYVPYTF
GAGTKLDIK

SEQ ID NO:20 is the amino acid sequence of the V_{L} of the S1-450-10 anti-FGFR4-Δ mAb

DIVMTQAAPSISVTPGESVSISCRSSESLLHSNGNTYLYWFLQRPGQSPQ
VLIYRMSSLASGVPDRFSGSGSGTVFILRISRVEAEDVGVYYCMQHLEYP
FTFGAGTKLELK

SEQ ID NO:21 is the amino acid sequence of the V_{L} of the Clone S1-509-7 anti-FGFR4-Δ mAb

DIVMTQSPSSLSASVGDRVTITCRASQDVSGNTYLDWYQQKPGKAPKLLI
YRMSRLPSGVPSRFSGSGSGTDFTLTISRLEPEDFAVYYCMQNMEFPFGS
GTKLDIK

SEQ ID NO:22 is the amino acid sequence of the V_{L} of the Clone S1-509-8 anti-FGFR4-Δ mAb

DIVMTQTPASLSASVGDRVTMTCRASQSVSGNTYLNWYQHKPGKAPKLLI
FKLSRLTGGVPSRFSGSGSGTDFTLTISRLEPEDFAVYYCMKHLEYKFGQ
GTKLEIK

Provided Below are the Amino Acid Sequences of Exemplary scFv Sequences of FGFR4-Δ Monoclonal Antibodies. CDR Sequences, as Determined by IMGT, are Shown in Bold; Linker Sequences are Shown in Italics.
SEQ ID NO:23 is the amino acid sequence of a scFv including the V_{H} and the V_{L} of the S3-357-7 anti-FGFR4-Δ mAb

DIQMTQSPSSLSASVGDRVTITCRASQNVGNTYLNWYQEKPGKAPKLLIY
KLSRLHSGVPSRFSGSGSGTDFTLTISRLEPEDFAVYYCQHSREVPWTFG
QGTKVEIKGSTSGGGSGGGSGGGGSSEVKLQESGAELVKPGVSVKISCKT
SGFTFSDYMDWVKQSHGKSLEWIGWINPNSGGTNYNQKFKGKATLTADKS
SSTAYMQLSSLTSEDSAVYYCARFGYDNSGAFFDYWGRGTLVTVSA

SEQ ID NO:24 is the amino acid sequence of a scFv including the V_{H} and the V_{L} of the S3-372-1 anti-FGFR4-Δ mAb

DIQMTQTTSSLSAVSGDRVTITCRASQDVAGNTYLNWYQHKPGKAPKLLV
YKLSSRPSGVPSRFSGSGSGTDFTLTISRLEPEDFAVYYCQQWTYVPYTF
GSGTKLEIKGSTSGGGSGGGSGGGGSSEVKLQQPGAELVRPGASVKISCK
ASGFTFSDYMHWVKQTHGKSLEWIGRIIGNAEGGYYNQKFKGKATLTADK
SSSTAYMQLSSLTSEDSAVYYCARDYFYGSTFYFDYWGQGTLVTVSA

SEQ ID NO:25 is the amino acid sequence of a scFv including the V_{H} and the V_{L} of the S3-420-5 anti-FGFR4-Δ mAb

DIVMTQSPSSLTASVGDRVTITCRASQSVSGNTYLNWYQQKPGKTPKLLI
YQISRLTGGVPSRFSGSGSGTDFTLTISRLEPEDFAVYYCMRHMDYPFGA
GTKLDIKGSTSGGGSGGGSGGGGSSEVKLQNAGAELVKPGTSVKISCKRS
GFTFSSYMDWVKQTHGKSLEWIGNITGNAGVAYYNQKFKGKATLTADKSS
STAYMQLSSLTSEDSAVYYCARVRDTSGYNYFFDYGRGTLVTVSAA

SEQ ID NO:26 is the amino acid sequence of a scFv including the V_{H} and the V_{L} of the S3-510-9 anti-FGFR4-Δ mAb

DIVMTQSPSSLSASVGDRITVTCRASQDVSGNTYLNWYQQKPGRAPKLLI
YYTSRLPSGVPSRFSGSGSGTDFTLTISRLEPEDFAVYYCQQFSYVPYTF
GSGTKLEIKGSTSGGGSGGGSGGGGSSEAKLEEAGAELVKPGATVRVTCK
GSGFTFSRYMDWVKQSHGKSLEWIGVITGNAGWSYYNQKFKGKATLTADK
SSSTAYMQLSSLTSEDSAVYYCARDYYGNSYYFDYWGQGTLVTVSA

SEQ ID NO:27 is the amino acid sequence of a scFv including the V_{H} and the V_{L} of the S3-553-7 anti-FGFR4-Δ mAb

DILMTQSPSSLSASVGDRVTITCRTSQSVSGNTYLNWYQQKPGKAPKFLI
YYTSRLTGGVPSRFSGSGSGTDFTLTISRLEPEDFAVYYCQHWSYVPYTF
GQGTKLEIKGSTSGGGSGGGSGGGGSSEVKLQKAGAELVKPGASVKISYR
NSGFTFSRYMDWVKQSHGKSLEWIANIKGNAGVGYYNQKFKGKATLTADK
SSSTAYMQLSSLTSEDSAVYYCARVRDTSGYNYFFDYWGQGTLVTVSA

SEQ ID NO:28 is the amino acid sequence of a scFv including the V_{H} and the V_{L} of the S1-37-6 anti-FGFR4-Δ mAb

DIVLTQSPSSLSASVGERVTITCRASQEVSGDTYLNWYQHKPGKTPLLLI
YQISRLPSGVPSRFSGSGSGTDFTLTISRLDPEDFAVYYCQQWSYVPFTF
GSGTKLDIKGSTSGGGSGGGSGGGGSSEVKLQQSGAELVKPGASVKISCK
ASGFTFSSYMDWVKQSHGKSLEWIGVITGNSGWGYYNQKFKGKATLTADK
SSSTAYMQLSSLTSEDSAVYYCARDYYGNSYHFDYWGRGTLVTVSA

SEQ ID NO:29 is the amino acid sequence of a scFv including the V_{H} and the V_{L} of the S1-37-7 anti-FGFR4-Δ mAb

DIVMTQAAPSISVTPGESVSISCRSSQDVSGHTYLNWYQQKPGKAPKLLI
YKLSRLPSGVPSRFSGSGSGTDFTLTISRMEPEDFAVYYCQQWSYMPFTF
GSGTKLEIKGSTSGGGSGGGSGGGGSSEVKLEESGGGLVQPGGSLKLSCV
ASGFSVNTHWMNWVRQSPTKGLEWVAEIRLKTNNYATHYAETVKGRFTIT
RDNSQSTVYLQMNSLRTEDTGIYYCTSKYNDYFDVWGSGTTVTVSS

SEQ ID NO:30 is the amino acid sequence of a scFv including the V_{H} and the V_{L} of the S1-450-4 anti-FGFR4-Δ mAb

DIVMTQSPSSLSASVGDRVTITCRASQSVSGNTYLNWYQRKPGKAPKLLI
YYTSRLTGGVPSRFSGSGSGTDFTLTISRLEPEDFAVYYCQHWSYVPYTF
GAGTKLDIKGSTSGGGSGGGSGGGGSSEVKLEESGGALVQPGGSMKLSCV
ASGYTVNSHWMSWVRQSPTKGLEWVAEIKLKSNKYASHFAESVKGRFTIT
RDNSQSTVYLQMNSLRAEDTGIYYCTSKFSDYFDVWGAGSTVTVAS

SEQ ID NO:31 is the amino acid sequence of a scFv including the V_{H} and the V_{L} of the S1-450-10 anti-FGFR4-Δ mAb

DIVMTQAAPSISVTPGESVSISCRSSESLLHSNGNTYLYWFLQRPGQSPQ
VLIYRMSSLASGVPDRFSGSGSGTVFILRISRVEAEDVGVYYCMQHLEYP
FTFGAGTKLELKGSTSGGGSGGGSGGGGSSEVKLEESGGGLVQPGGSMKL
SCVASGFTVNTHWMNWVRQSPEKGLEWVAEIRLKSNNYATHYAESVKGRF
TISRDNSQSTVYLQMNNLRTEDTGIYYCTSKYNWYFDVWGAGTTVTVSS

SEQ ID NO:32 is the amino acid sequence of a scFv including the V_{H} and the V_{L} of the S1-509-7 anti-FGFR4-Δ mAb

DIVMTQSPSSLSASVGDRVTITCRASQDVSGNTYLDWYQQKPGKAPKLLI
YRMSRLPSGVPSRFSGSGSGTDFTLTISRLEPEDFAVYYCMQNMEFPFGS
GTKLDIKGSTSGGGSGGGSGGGGSSEVKLEESGGGLVQPGGSMKLTCTAS
GYSVNTHFLNWVRQSPTKGLEWVAEIRLRSNDYATHFAESVKGRFTITRD
GSQSTVYLQMNHLRTEDTGIYYCTSRYSDYFDVWGAGSTVTVAS

SEQ ID NO:33 is the amino acid sequence of a scFv including the V_{H} and the V_{L} of the S1-509-8 anti-FGFR4-Δ mAb

DIVMTQTPASLSASVGDRVTMTCRASQSVSGNTYLNWYQHKPGKAPKLLI
FKLSRLTGGVPSRFSGSGSGTDFTLTISRLEPEDFAVYYCMKHLEYKFGQ
GTKLEIKGSTSGGGSGGGSGGGGSSEVKLEDSGGGLVQPGGSMKLSCIAS
GFAVNTYWMNWVRQSPTKGLEWVPEIRVKSNGYATHYAEAVKGRFTITRD
GSQSTVFLQMNNLRTEDTGIYYCTTKFSDYFDVWGVGTTVTVAS

### Provided Below are the Amino Acid Sequences of Exemplary Peptide Linker and Signaling Domain Sequences for CAR Construction

SEQ ID NO:34 is the amino acid sequence of an exemplary peptide linker for antibody-based CARs

GSTSGGGSGGGSGGGGSS

SEQ ID NO:35 is the amino acid sequence of an exemplary signaling peptide

MLLLVTSLLLCELPHPAFLLIP

SEQ ID NO:36 is the amino acid sequence of exemplary CD28 transmembrane and signaling domains

IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRS

SEQ ID NO:37 is the amino acid sequence of an exemplary CD3& T cell activating domain

RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

### Provided Below are the Nucleotide Sequences of FGFR4-Δ Specific Primers and Probe

SEQ ID NO:38 is the nucleotide sequences of FGFR4-Δ Forward Primer

TGGAGGCCTCTGAGGAAGT

SEQ ID NO:39 is the nucleotide sequences of FGFR4-Δ Reverse Primer

GGGTCCTCATCATCGTTGCT

SEQ ID NO:40 is the nucleotide sequence of FGFR4-Δ Probe

CAAGGAGTCAAGCTCC

SEQ ID NO:41 the amino acid sequence of FGFR4-Δ

MRLLLALLGVLLSVPGPPVLSLEASEEVELDSLTSSNDDEDPKSHRDPSN
RHSYPQQAPYWTHPQRMEKKLHAVPAGNTVKFRCPAAGNPTPTIRWLKDG
QAFHGENRIGGIRLRHQHWSLVMESVVPSDRGTYTCLVENAVGSIRYNYL
LDVLERSPHRPILQAGLPANTTAVVGSDVELLCKVYSDAQPHIQWLKHIV
INGSSFGADGFPYVQVLKTADINSSEVEVLYLRNVSAEDAGEYTCLAGNS
IGLSYQSAWLTVLPEEDPTWTAAAPEARYTDIILYASGSLALAVLLLLAG
LYRGQALHGRHPRPPATVQKLSRFPLARQFSLESGSSGKSSSSLVRGVRL
SSSGPALLAGLVSLDLPLDPLWEFPRDRLVLGKPLGEGCFGQVVRAEAFG
MDPARPDQASTVAVKMLKDNASDKDLADLVSEMEVMKLIGRHKNIINLLG
VCTQEGPLYVIVECAAKGNLREFLRARRPPGPDLSPDGPRSSEGPLSFPV
LVSCAYQVARGMQYLESRKCIHRDLAARNVLVTEDNVMKIADFGLARGVH
HIDYYKKTSNGRLPVKWMAPEALFDRVYTHQSDVWSFGILLWEIFTLGGS
PYPGIPVEELFSLLREGHRMDRPPHCPPELYGLMRECWHAAPSQRPTFKQ
LVEALDKVLLAVSEEYLDLRLTFGPYSPSGGDASSTCSSSDSVFSHDPLP
LGSSSFPFGSGVQT

SEQ ID NO:42 the polynucleotide sequence encoding FGFR4-Δ

ATGCGGCTGCTGCTGGCCCTGTTGGGGGTCCTGCTGAGTGTGCCTGGGCC
TCCAGTCTTGTCCCTGGAGGCCTCTGAGGAAGTGGAGCTTGACTCCTTGA
CCTCCAGCAACGATGATGAGGACCCCAAGTCCCATAGGGACCCCTCGAAT
AGGCACAGTTACCCCCAGCAAGCACCCTACTGGACACACCCCCAGCGCAT
GGAGAAGAAACTGCATGCAGTACCTGCGGGGAACACCGTCAAGTTCCGCT
GTCCAGCTGCAGGCAACCCCACGCCCACCATCCGCTGGCTTAAGGATGGA
CAGGCCTTTCATGGGGAGAACCGCATTGGAGGCATTCGGCTGCGCCATCA
GCACTGGAGTCTCGTGATGGAGAGCGTGGTGCCCTCGGACCGCGGCACAT
ACACCTGCCTGGTAGAGAACGCTGTGGGCAGCATCCGCTATAACTACCTG
CTAGATGTGCTGGAGCGGTCCCCGCACCGGCCCATCCTGCAGGCCGGGCT
CCCGGCCAACACCACAGCCGTGGTGGGCAGCGACGTGGAGCTGCTGTGCA
AGGTGTACAGCGATGCCCAGCCCCACATCCAGTGGCTGAAGCACATCGTC
ATCAACGGCAGCAGCTTCGGAGCCGACGGTTTCCCCTATGTGCAAGTCCT
AAAGACTGCAGACATCAATAGCTCAGAGGTGGAGGTCCTGTACCTGCGGA
ACGTGTCAGCCGAGGACGCAGGCGAGTACACCTGCCTCGCAGGCAATTCC
ATCGGCCTCTCCTACCAGTCTGCCTGGCTCACGGTGCTGCCAGAGGAGGA
CCCCACATGGACCGCAGCAGCGCCCGAGGCCAGGTATACGGACATCATCC
TGTACGCGTCGGGCTCCCTGGCCTTGGCTGTGCTCCTGCTGCTGGCCGGG
CTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGT
CAGGCTCTTCCGGCAAGTCAAGCTCATCCCTGGTACGAGGCGTGCGTCTC
TCCTCCAGCGGCCCCGCCTTGCTCGCCGGCCTCGTGAGTCTAGATCTACC
TCTCGACCCACTATGGGAGTTCCCCCGGGACAGGCTGGTGCTTGGGAAGC
CCCTAGGCGAGGGCTGCTTTGGCCAGGTAGTACGTGCAGAGGCCTTTGGC
ATGGACCCTGCCCGGCCTGACCAAGCCAGCACTGTGGCCGTCAAGATGCT
CAAAGACAACGCCTCTGACAAGGACCTGGCCGACCTGGTCTCGGAGATGG
AGGTGATGAAGCTGATCGGCCGACACAAGAACATCATCAACCTGCTTGGT
GTCTGCACCCAGGAAGGGCCCCTGTACGTGATCGTGGAGTGCGCCGCCAA
GGGAAACCTGCGGGAGTTCCTGCGGGCCCGGCGCCCCCCAGGCCCCGACC
TCAGCCCCGACGGTCCTCGGAGCAGTGAGGGGCCGCTCTCCTTCCCAGTC
CTGGTCTCCTGCGCCTACCAGGTGGCCCGAGGCATGCAGTATCTGGAGTC
CCGGAAGTGTATCCACCGGGACCTGGCTGCCCGCAATGTGCTGGTGACTG
AGGACAATGTGATGAAGATTGCTGACTTTGGGCTGGCCCGCGGCGTCCAC
CACATTGACTACTATAAGAAAACCAGCAACGGCCGCCTGCCTGTGAAGTG
GATGGCGCCCGAGGCCTTGTTTGACCGGGTGTACACACACCAGAGTGACG
TGTGGTCTTTTGGGATCCTGCTATGGGAGATCTTCACCCTCGGGGGCTCC
CCGTATCCTGGCATCCCGGTGGAGGAGCTGTTCTCGCTGCTGCGGGAGGG
ACATCGGATGGACCGACCCCCACACTGCCCCCCAGAGCTGTACGGGCTGA
TGCGTGAGTGCTGGCACGCAGCGCCCTCCCAGAGGCCTACCTTCAAGCAG
CTGGTGGAGGCGCTGGACAAGGTCCTGCTGGCCGTCTCTGAGGAGTACCT
CGACCTCCGCCTGACCTTCGGACCCTATTCCCCCTCTGGTGGGGACGCCA
GCAGCACCTGCTCCTCCAGCGATTCTGTCTTCAGCCACGACCCCCTGCCA
TTGGGATCCAGCTCCTTCCCCTTCGGGTCTGGGGTGCAGACATGA

TABLE 1
CDR amino acid sequences of anti-FGFR4-Δ monoclonal antibodies based on the
IMGT numbering system

| | | IMGT | | |
|---|---|---|---|---|
| Description | Clone Name | CDRI | CDR2 | CDR3 |
| VH | S3-357-7 | GFTFSDY | INPNSGGT | ARFGYDNSGAFFDY |
| VH | S3-372-1 | GFTFSDY | IIGNAEGG | ARDYFYGSTFYFDY |
| VH | S3-420-5 | GFTFSSY | ITGNAGVA | ARVRDTSGYNYFFDY |
| VH | S3-510-9 | GFTFSRY | ITGNAGWS | ARDYYGNSYYFDY |
| VH | S3-553-7 | GFTFSRY | IKGNAGVG | ARVRDTSGYNYFFDY |
| VH | S1-37-6 | GFTFSSY | ITGNSGWG | ARDYYGNSYHFDY |
| VH | S1-37-7 | GFSVNTHW | IRLKTNNYAT | TSKYNDYFDV |
| VH | S1-450-4 | GYTVNSHW | IKLKSNKYA | TSKFSDYFDV |
| VH | S1-450-10 | GFTVNTHW | IRLKSNNYAT | TSKYNWYFDV |
| VH | S1-509-7 | GYSVNTHF | IRLRSNDYAT | TSRYSDYFDV |
| VH | S1-509-8 | GFAVNTYW | IRVKSNGYAT | TTKFSDYFDV |
| VL | S3-357-7 | QNVGNTY | KLS | QHSREVPWT |
| VL | S3-372-1 | QDVAGNTY | KLS | QQWTYVPYT |
| VL | S3-420-5 | QSVSGNTY | QIS | MRHMDYP |
| VL | S3-510-9 | QDVSGNTY | YTS | QQFSYVPYT |
| VL | S3-553-7 | QSVSGNTY | YTS | QHWSYVPYT |
| VL | S1-37-6 | QEVSGDTY | QIS | QQWSYVPFT |
| VL | S1-37-7 | QDVSGHTY | KLS | QQWSYMPFT |
| VL | S1-450-4 | QSVSGNTY | YTS | QHWSYVPYT |
| VL | S1-450-10 | ESLLHSNGNTY | RMS | MQHLEYP |
| VL | S1-509-7 | QDVSGNTY | RMS | MQNMEFP |
| VL | S1-509-8 | QSVSGNTY | KLS | MKHLEYK |

TABLE 2
CDR amino acid sequences of anti-FGFR4-Δ monoclonal antibodies based on the
KABAT numbering system

| | | KABAT | | |
|---|---|---|---|---|
| Description | Clone Name | CDR1 | CDR2 | CDR3 |
| VH | S3-357-7 | DYMD | WINPNSGGTNYNQKFKG | FGYDNSGAFFDY |
| VH | S3-372-3 | DYMH | RIIGNAEGGYYNQKFKG | DYFYGSTFYFDY |
| VH | S3-420-5 | SYND | NITGNAGVAYYNQKFKG | VRDTSGYNYFFD |
| VH | S3-510-9 | RYMD | VITGNAGWSYYNQKFKG | DYYGNSYYFDY |
| VH | S3-553-7 | FTFSRYMD | NIKGNAGVGYYNQKFKS | VRDTSGYNYFFDY |
| VH | S1-37-6 | SYMD | VITGNSGWGYYNQKFKG | DYYGNSYHFDY |
| VH | S1-37-7 | THWMN | EIRLKTNNYATHYAETVKG | KYNDYFDV |
| VH | S1-450-4 | SHWMS | EIKLKSNKYASHFAESVKG | KFSDYFDV |
| VH | S1-450-10 | THWMN | EIRLKSNNYATHYAESVKG | KYNWYFDV |
| VH | S1-509-7 | THFLN | EIRLRSNDYATHFAESVKG | RYSDYFDV |
| VH | S1-509-8 | TYWMN | EIRVKSNGYATHYAEAVKG | KFSDYFDV |
| VH | S3-357-7 | RASQNVGNTYLN | KLSRLHS | QHSREVPWT |
| VL | S3-372-1 | RASQDVAGNTYLN | KLSSRPS | QQWTYVPYT |
| VL | S3-420-5 | RASQSVSGNTYLN | QISRLTG | MRHMDYP |
| VL | S3-510-9 | RASQDVSGNTYLN | YTSRLPS | QQFSYVPYT |
| VL | S3-553-7 | RTSQSVSGNTYLN | YTSRLTG | QHWSYVPYT |
| VL | S1-37-6 | RASQEVSGDTYLN | QISRLPS | QQWSYVPFT |
| VL | S1-37-7 | RSSQDVSGHTYLN | KLSRLPS | QQWSYMPFT |
| VL | S1-450-4 | RASQSVSGNTYLN | YTSRLTG | QHWSYVPYT |
| VL | S1-450-10 | RSSESLLHSNGNTYLY | RMSSLAS | MQHLEYPFT |
| VL | S1-509-7 | RASQDVSGNTYLD | RMSRLPS | MQNMEFP |
| VL | S1-509-8 | RASQSVSGNTYLN | KLSRLTG | MKHLEYK |

1. An antibody construct that specifically binds FGFR4-Δ and comprises antibody heavy chain and light chain complementarity determining regions (CDRs) having the amino acid sequences set forth in Table 1 or Table 2 according to the IMGT or KABAT numbering system, respectively.

2. The antibody construct of claim 1, comprising antibody heavy chain variable region (V_{H}) and light chain variable region (V_{L}), each comprising the amino acid sequence set forth in SEQ ID NOs: 1 and 12, SEQ ID NOs: 2 and 13, SEQ ID NOs: 3 and 14, SEQ ID NOs: 4 and 15, SEQ ID NOs: 5 and 16, SEQ ID NOs: 6 and 17, SEQ ID NOs: 7 and 18, SEQ ID NOs: 8 and 19, SEQ ID NOs: 9 and 20, SEQ ID NOs: 10 and 21, SEQ ID NOs: 11 and 22, respectively.

3. The antibody construct of claim 1, which is an anti-FGFR4-Δ single chain variable fragment (scFv).

4. The antibody construct of claim 1, comprising the amino acid sequence set forth in SEQ ID NO:23, 24, 25, 26, 27, 28, 29, 30, 31, 32, or 33.

5. The antibody construct of claim 1, which is a chimeric antigen receptor (CAR) comprising, from its N-terminus, an anti-FGFR4-Δ scFv, a CD28 transmembrane domain, and a CD3ζ T cell activating domain.

6. The antibody construct of claim 5, comprising the amino acid sequences set forth in SEQ ID NOs: 23, 36, and 37.

7. A nucleic acid comprising a polynucleotide sequence encoding the antibody construct claim 1.

8. The nucleic acid of claim 7, which is an expression cassette comprising a promoter operably linked to the polynucleotide sequence.

9. The nucleic acid of claim 7, which is a plasmid or a viral vector.

10. A host cell comprising the nucleic acid of claim 7.

11. The host cell of claim 10, which is a T cell or natural killer (NK) cell.

12. The host cell of claim 11, wherein the antibody construct is a CAR.

13. The host cell of claim 12, which is a T cell expressing a CAR comprising the amino acid sequences set forth in SEQ ID NOs: 23, 36, and 37.

14. A conjugate comprising the antibody construct of claim 1 and a solid support, a detectable moiety, or a therapeutic agent.

15. The conjugate of claim 14, wherein the antibody construct is an anti-FGFR4-Δ scFv, and wherein the therapeutic agent comprises an anti-cancer therapeutic agent.

16. A composition comprising the antibody construct of claim 1.

17. A method for killing a cell expressing FGFR4-Δ, comprising contacting cancer cells with a composition comprising an adequate number of T cells expressing the antibody construct of claim 5.

18. The method of claim 17, wherein the cell expressing FGFR4-Δ is a cancer cell.

19. The method of claim 18, wherein the cancer cell is a hepatocellular carcinoma (HCC) cell.

20. The method of claim 18, wherein the cancer cell is located in a patient's body.

21. The method of claim 17, further comprising, prior to the contacting step, (i) isolating T cells from a patient; (ii) transfecting the T cells with the nucleic acid encoding the antibody construct; (iii) cultivating the T cells ex vivo to expanding T cells expressing a CAR comprising, from its N-terminus, an anti-FGFR4-Δ scFv, a CD28 transmembrane domain, and a CD3ζT cell activating domain, and (iv) administering the expanded T cells back into the patient's body.

22. The method of claim 21, wherein the CAR comprises the amino acid sequences set forth in SEQ ID NOs: 23, 36, and 37.

23. The method of claim 21, wherein step (iv) comprises subcutaneous, intravenous, intramuscular, intraperitoneal, or intratumoral injection.

24. A method for treating a disease involving FGFR4-Δ expression in a patient, comprising administering to the patient with a composition comprising an effective amount of a nucleic acid encoding the antibody construct of claim 5.

25. The method of claim 24, wherein the disease is a cancer.

26. The method of claim 25, wherein the cancer is hepatocellular carcinoma (HCC).

27. The method of claim 24, wherein the nucleic acid is a viral vector.

28. The method of claim 24, wherein the composition comprises the nucleic acid formulated with lipid nanoparticles.

29. The method of claim 24, wherein the CAR comprises the amino acid sequences set forth in SEQ ID NOs: 23, 36, and 37.

30. The method of claim 24, wherein the administering comprises subcutaneous, intravenous, intramuscular, intraperitoneal, or intratumoral injection.

31. A kit for treating cancer, comprising
(1) a first container containing a first composition comprising an adequate number of T cells expressing a CAR comprising, from its N-terminus, an anti-FGFR4-Δ scFv, a CD28 transmembrane domain, and a CD3ζ T cell activating domain; and
(2) a second container containing a second composition comprising an effective amount of another anti-cancer therapeutic agent.

32. The kit of claim 31, wherein the cancer is hepatocellular carcinoma.

33. The kit of claim 31, wherein the CAR comprises the amino acid sequences set forth in SEQ ID NOs:23, 36, and 37.

34. The kit of claim 31, wherein the first composition is formulated for injection.

35. The kit of claim 34, wherein the injection is subcutaneous, intravenous, intramuscular, intraperitoneal, or intratumoral injection.

36. The kit of claim 31, further comprising an instruction manual for using the kit.

37. A polypeptide comprising the amino acid sequence set forth in SEQ ID NO:41, optionally conjugated with a heterologous moiety.

38. A nucleic acid comprising a polynucleotide sequence encoding a polypeptide comprising the amino acid sequence set forth in SEQ ID NO:41, optionally further comprising a heterologous nucleotide sequence.

39. The nucleic acid of claim 37, which is an expression cassette comprising a promoter operably linked to the polynucleotide sequence.

40. The nucleic acid of claim 37, which is a plasmid or a viral vector.

41. A host cell comprising the nucleic acid of claim 31.

42. A composition comprising the polypeptide of claim 37.


SEQUENCE LISTING (42 sequences)

SEQ ID NO: 1 - protein, 120 aa, synthetic construct
  REGION 1..120: note = Description of sequence: Monoclonal antibody component, particularly, amino acid sequence of the VH of the S3-357-7 anti-FGFR4-Delta mAb
EVKLQESGAELVKPGVSVKISCKTSGFTFSDYMDWVKQSHGKSLEWIGWINPNSGGTNYNQKFKGKATLTADKSSSTAYMQLSSLTSEDSAVYYCARFGYDNSGAFFDYWGRGTLVTVSA

SEQ ID NO: 2 - protein, 120 aa, synthetic construct
  REGION 1..120: note = Description of sequence: Monoclonal antibody component, particularly, amino acid sequence of the VH of the S3-372-1 anti-FGFR4-Delta mAb
EVKLQQPGAELVRPGASVKISCKASGFTFSDYMHWVKQTHGKSLEWIGRIIGNAEGGYYNQKFKGKATLTADKSSSTAYMQLSSLTSEDSAVYYCARDYFYGSTFYFDYWGQGTLVTVSA

SEQ ID NO: 3 - protein, 121 aa, synthetic construct
  REGION 1..121: note = Description of sequence: Monoclonal antibody component, particularly, amino acid sequence of the VH of the S3-420-5 anti-FGFR4-Delta mAb
EVKLQNAGAELVKPGTSVKISCKRSGFTFSSYMDWVKQTHGKSLEWIGNITGNAGVAYYNQKFKGKATLTADKSSSTAYMQLSSLTSEDSAVYYCARVRDTSGYNYFFDYGRGTLVTVSAA

SEQ ID NO: 4 - protein, 119 aa, synthetic construct
  REGION 1..119: note = Description of sequence: Monoclonal antibody component, particularly, amino acid sequence of the VH of the S3-510-9 anti-FGFR4-Delta mAb
EAKLEEAGAELVKPGATVRVTCKGSGFTFSRYMDWVKQSHGKSLEWIGVITGNAGWSYYNQKFKGKATLTADKSSSTAYMQLSSLTSEDSAVYYCARDYYGNSYYFDYWGQGTLVTVSA

SEQ ID NO: 5 - protein, 121 aa, synthetic construct
  REGION 1..121: note = Description of sequence: Monoclonal antibody component, particularly, amino acid sequence of the VH of the S3-553-7 anti-FGFR4-Delta mAb
EVKLQKAGAELVKPGASVKISYRNSGFTFSRYMDWVKQSHGKSLEWIANIKGNAGVGYYNQKFKGKATLTADKSSSTAYMQLSSLTSEDSAVYYCARVRDTSGYNYFFDYWGQGTLVTVSA

SEQ ID NO: 6 - protein, 119 aa, synthetic construct
  REGION 1..119: note = Description of sequence: Monoclonal antibody component, particularly, amino acid sequence of the VH of the S1-37-6 anti-FGFR4-Delta mAb
EVKLQQSGAELVKPGASVKISCKASGFTFSSYMDWVKQSHGKSLEWIGVITGNSGWGYYNQKFKGKATLTADKSSSTAYMQLSSLTSEDSAVYYCARDYYGNSYHFDYWGRGTLVTVSA

SEQ ID NO: 7 - protein, 119 aa, synthetic construct
  REGION 1..119: note = Description of sequence: Monoclonal antibody component, particularly, amino acid sequence of the VH of the Clone S1-37-7 anti-FGFR4-Delta mAb
EVKLEESGGGLVQPGGSLKLSCVASGFSVNTHWMNWVRQSPTKGLEWVAEIRLKTNNYATHYAETVKGRFTITRDNSQSTVYLQMNSLRTEDTGIYYCTSKYNDYFDVWGSGTTVTVSS

SEQ ID NO: 8 - protein, 119 aa, synthetic construct
  REGION 1..119: note = Description of sequence: Monoclonal antibody component, particularly, amino acid sequence of the VH of the Clone S1-450-4 anti-FGFR4-Delta mAb
EVKLEESGGALVQPGGSMKLSCVASGYTVNSHWMSWVRQSPTKGLEWVAEIKLKSNKYASHFAESVKGRFTITRDNSQSTVYLQMNSLRAEDTGIYYCTSKFSDYFDVWGAGSTVTVAS

SEQ ID NO: 9 - protein, 119 aa, synthetic construct
  REGION 1..119: note = Description of sequence: Monoclonal antibody component, particularly, amino acid sequence of the VH of the S1-450-10 anti-FGFR4-Delta mAb
EVKLEESGGGLVQPGGSMKLSCVASGFTVNTHWMNWVRQSPEKGLEWVAEIRLKSNNYATHYAESVKGRFTISRDNSQSTVYLQMNNLRTEDTGIYYCTSKYNWYFDVWGAGTTVTVSS

SEQ ID NO: 10 - protein, 119 aa, synthetic construct
  REGION 1..119: note = Description of sequence: Monoclonal antibody component, particularly, amino acid sequence of the VH of the Clone S1-509-7 anti-FGFR4-Delta mAb
EVKLEESGGGLVQPGGSMKLTCTASGYSVNTHFLNWVRQSPTKGLEWVAEIRLRSNDYATHFAESVKGRFTITRDGSQSTVYLQMNHLRTEDTGIYYCTSRYSDYFDVWGAGSTVTVAS

SEQ ID NO: 11 - protein, 119 aa, synthetic construct
  REGION 1..119: note = Description of sequence: Monoclonal antibody component, particularly, amino acid sequence of the VH of the Clone S1-509-8 anti-FGFR4-Delta mAb
EVKLEDSGGGLVQPGGSMKLSCIASGFAVNTYWMNWVRQSPTKGLEWVPEIRVKSNGYATHYAEAVKGRFTITRDGSQSTVFLQMNNLRTEDTGIYYCTTKFSDYFDVWGVGTTVTVAS

SEQ ID NO: 12 - protein, 108 aa, synthetic construct
  REGION 1..108: note = Description of sequence: Monoclonal antibody component, particularly, amino acid sequence of the VL of the S3-357-7 anti-FGFR4-Delta mAb
DIQMTQSPSSLSASVGDRVTITCRASQNVGNTYLNWYQEKPGKAPKLLIYKLSRLHSGVPSRFSGSGSGTDFTLTISRLEPEDFAVYYCQHSREVPWTFGQGTKVEIK

SEQ ID NO: 13 - protein, 109 aa, synthetic construct
  REGION 1..109: note = Description of sequence: Monoclonal antibody component, particularly, amino sequence of the VL of the S3-372-1 anti-FGFR4-Delta mAb
DIQMTQTTSSLSAVSGDRVTITCRASQDVAGNTYLNWYQHKPGKAPKLLVYKLSSRPSGVPSRFSGSGSGTDFTLTISRLEPEDFAVYYCQQWTYVPYTFGSGTKLEIK

SEQ ID NO: 14 - protein, 107 aa, synthetic construct
  REGION 1..107: note = Description of sequence: Monoclonal antibody component, particularly, amino acid sequence of the VL of the S3-420-5 anti-FGFR4-Delta mAb
DIVMTQSPSSLTASVGDRVTITCRASQSVSGNTYLNWYQQKPGKTPKLLIYQISRLTGGVPSRFSGSGSGTDFTLTISRLEPEDFAVYYCMRHMDYPFGAGTKLDIK

SEQ ID NO: 15 - protein, 109 aa, synthetic construct
  REGION 1..109: note = Description of sequence: Monoclonal antibody component, particularly, amino acid sequence of the VL of the S3-510-9 anti-FGFR4-Delta mAb
DIVMTQSPSSLSASVGDRITVTCRASQDVSGNTYLNWYQQKPGRAPKLLIYYTSRLPSGVPSRFSGSGSGTDFTLTISRLEPEDFAVYYCQQFSYVPYTFGSGTKLEIK

SEQ ID NO: 16 - protein, 109 aa, synthetic construct
  REGION 1..109: note = Description of sequence: Monoclonal antibody component, particularly, amino acid sequence of the VL of the S3-553-7 anti-FGFR4-Delta mAb
DILMTQSPSSLSASVGDRVTITCRTSQSVSGNTYLNWYQQKPGKAPKFLIYYTSRLTGGVPSRFSGSGSGTDFTLTISRLEPEDFAVYYCQHWSYVPYTFGQGTKLEIK

SEQ ID NO: 17 - protein, 109 aa, synthetic construct
  REGION 1..109: note = Description of sequence: Monoclonal antibody component, particularly, amino acid sequence of the VL of the S1-37-6 anti-FGFR4-Delta mAb
DIVLTQSPSSLSASVGERVTITCRASQEVSGDTYLNWYQHKPGKTPLLLIYQISRLPSGVPSRFSGSGSGTDFTLTISRLDPEDFAVYYCQQWSYVPFTFGSGTKLDIK

SEQ ID NO: 18 - protein, 109 aa, synthetic construct
  REGION 1..109: note = Description of sequence: Monoclonal antibody component, particularly, amino acid sequence of the VL of the Clone S1-37-7 anti-FGFR4-Delta mAb
DIVMTQAAPSISVTPGESVSISCRSSQDVSGHTYLNWYQQKPGKAPKLLIYKLSRLPSGVPSRFSGSGSGTDFTLTISRMEPEDFAVYYCQQWSYMPFTFGSGTKLEIK

SEQ ID NO: 19 - protein, 109 aa, synthetic construct
  REGION 1..109: note = Description of sequence: Monoclonal antibody component, particularly, amino acid sequence of the VL of the Clone S1-450-4 anti-FGFR4-Delta mAb
DIVMTQSPSSLSASVGDRVTITCRASQSVSGNTYLNWYQRKPGKAPKLLIYYTSRLTGGVPSRFSGSGSGTDFTLTISRLEPEDFAVYYCQHWSYVPYTFGAGTKLDIK

SEQ ID NO: 20 - protein, 112 aa, synthetic construct
  REGION 1..112: note = Description of sequence: Monoclonal antibody component, particularly, amino acid sequence of the VL of the S1-450-10 anti-FGFR4-Delta mAb
DIVMTQAAPSISVTPGESVSISCRSSESLLHSNGNTYLYWFLQRPGQSPQVLIYRMSSLASGVPDRFSGSGSGTVFILRISRVEAEDVGVYYCMQHLEYPFTFGAGTKLELK

SEQ ID NO: 21 - protein, 107 aa, synthetic construct
  REGION 1..107: note = Description of sequence: Monoclonal antibody component, particularly, amino acid sequence of the VL of the Clone S1-509-7 anti-FGFR4-Delta mAb
DIVMTQSPSSLSASVGDRVTITCRASQDVSGNTYLDWYQQKPGKAPKLLIYRMSRLPSGVPSRFSGSGSGTDFTLTISRLEPEDFAVYYCMQNMEFPFGSGTKLDIK

SEQ ID NO: 22 - protein, 107 aa, synthetic construct
  REGION 1..107: note = Description of sequence: Monoclonal antibody component, particularly, amino acid sequence of the VL of the Clone S1-509-8 anti-FGFR4-Delta mAb
DIVMTQTPASLSASVGDRVTMTCRASQSVSGNTYLNWYQHKPGKAPKLLIFKLSRLTGGVPSRFSGSGSGTDFTLTISRLEPEDFAVYYCMKHLEYKFGQGTKLEIK

SEQ ID NO: 23 - protein, 246 aa, synthetic construct
  REGION 1..246: note = Description of sequence: Monoclonal antibody component, particularly, amino acid sequence of a scFv including the VH and the VL of the S3-357-7 anti-FGFR4-Delta mAb
DIQMTQSPSSLSASVGDRVTITCRASQNVGNTYLNWYQEKPGKAPKLLIYKLSRLHSGVPSRFSGSGSGTDFTLTISRLEPEDFAVYYCQHSREVPWTFGQGTKVEIKGSTSGGGSGGGSGGGGSSEVKLQESGAELVKPGVSVKISCKTSGFTFSDYMDWVKQSHGKSLEWIGWINPNSGGTNYNQKFKGKATLTADKSSSTAYMQLSSLTSEDSAVYYCARFGYDNSGAFFDYWGRGTLVTVSA

SEQ ID NO: 24 - protein, 247 aa, synthetic construct
  REGION 1..247: note = Description of sequence: Monoclonal antibody component, particularly, amino acid sequence of a scFv including the VH and the VL of the S3-372-1 anti-FGFR4-Delta mAb
DIQMTQTTSSLSAVSGDRVTITCRASQDVAGNTYLNWYQHKPGKAPKLLVYKLSSRPSGVPSRFSGSGSGTDFTLTISRLEPEDFAVYYCQQWTYVPYTFGSGTKLEIKGSTSGGGSGGGSGGGGSSEVKLQQPGAELVRPGASVKISCKASGFTFSDYMHWVKQTHGKSLEWIGRIIGNAEGGYYNQKFKGKATLTADKSSSTAYMQLSSLTSEDSAVYYCARDYFYGSTFYFDYWGQGTLVTVSA

SEQ ID NO: 25 - protein, 246 aa, synthetic construct
  REGION 1..246: note = Description of sequence: Monoclonal antibody component, particularly, amino acid sequence of a scFv including the VH and the VL of the S3-420-5 anti-FGFR4-Delta mAb
DIVMTQSPSSLTASVGDRVTITCRASQSVSGNTYLNWYQQKPGKTPKLLIYQISRLTGGVPSRFSGSGSGTDFTLTISRLEPEDFAVYYCMRHMDYPFGAGTKLDIKGSTSGGGSGGGSGGGGSSEVKLQNAGAELVKPGTSVKISCKRSGFTFSSYMDWVKQTHGKSLEWIGNITGNAGVAYYNQKFKGKATLTADKSSSTAYMQLSSLTSEDSAVYYCARVRDTSGYNYFFDYGRGTLVTVSAA

SEQ ID NO: 26 - protein, 246 aa, synthetic construct
  REGION 1..246: note = Description of sequence: Monoclonal antibody component, particularly, amino acid sequence of a scFv including the VH and the VL of the S3-510-9 anti-FGFR4-Delta mAb
DIVMTQSPSSLSASVGDRITVTCRASQDVSGNTYLNWYQQKPGRAPKLLIYYTSRLPSGVPSRFSGSGSGTDFTLTISRLEPEDFAVYYCQQFSYVPYTFGSGTKLEIKGSTSGGGSGGGSGGGGSSEAKLEEAGAELVKPGATVRVTCKGSGFTFSRYMDWVKQSHGKSLEWIGVITGNAGWSYYNQKFKGKATLTADKSSSTAYMQLSSLTSEDSAVYYCARDYYGNSYYFDYWGQGTLVTVSA

SEQ ID NO: 27 - protein, 248 aa, synthetic construct
  REGION 1..248: note = Description of sequence: Monoclonal antibody component, particularly, amino acid sequence of a scFv including the VH and the VL of the S3-553-7 anti-FGFR4-Delta mAb
DILMTQSPSSLSASVGDRVTITCRTSQSVSGNTYLNWYQQKPGKAPKFLIYYTSRLTGGVPSRFSGSGSGTDFTLTISRLEPEDFAVYYCQHWSYVPYTFGQGTKLEIKGSTSGGGSGGGSGGGGSSEVKLQKAGAELVKPGASVKISYRNSGFTFSRYMDWVKQSHGKSLEWIANIKGNAGVGYYNQKFKGKATLTADKSSSTAYMQLSSLTSEDSAVYYCARVRDTSGYNYFFDYWGQGTLVTVSA

SEQ ID NO: 28 - protein, 246 aa, synthetic construct
  REGION 1..246: note = Description of sequence: Monoclonal antibody component, particularly, amino acid sequence of a scFv including the VH and the VL of the S1-37-6 anti-FGFR4-Delta mAb
DIVLTQSPSSLSASVGERVTITCRASQEVSGDTYLNWYQHKPGKTPLLLIYQISRLPSGVPSRFSGSGSGTDFTLTISRLDPEDFAVYYCQQWSYVPFTFGSGTKLDIKGSTSGGGSGGGSGGGGSSEVKLQQSGAELVKPGASVKISCKASGFTFSSYMDWVKQSHGKSLEWIGVITGNSGWGYYNQKFKGKATLTADKSSSTAYMQLSSLTSEDSAVYYCARDYYGNSYHFDYWGRGTLVTVSA

SEQ ID NO: 29 - protein, 246 aa, synthetic construct
  REGION 1..246: note = Description of sequence: Monoclonal antibody component, particularly, amino acid sequence of a scFv including the VH and the VL of the S1-37-7 anti-FGFR4-Delta mAb
DIVMTQAAPSISVTPGESVSISCRSSQDVSGHTYLNWYQQKPGKAPKLLIYKLSRLPSGVPSRFSGSGSGTDFTLTISRMEPEDFAVYYCQQWSYMPFTFGSGTKLEIKGSTSGGGSGGGSGGGGSSEVKLEESGGGLVQPGGSLKLSCVASGFSVNTHWMNWVRQSPTKGLEWVAEIRLKTNNYATHYAETVKGRFTITRDNSQSTVYLQMNSLRTEDTGIYYCTSKYNDYFDVWGSGTTVTVSS

SEQ ID NO: 30 - protein, 246 aa, synthetic construct
  REGION 1..246: note = Description of sequence: Monoclonal antibody component, particularly, amino acid sequence of a scFv including the VH and the VL of the S1-450-4 anti-FGFR4-Delta mAb
DIVMTQSPSSLSASVGDRVTITCRASQSVSGNTYLNWYQRKPGKAPKLLIYYTSRLTGGVPSRFSGSGSGTDFTLTISRLEPEDFAVYYCQHWSYVPYTFGAGTKLDIKGSTSGGGSGGGSGGGGSSEVKLEESGGALVQPGGSMKLSCVASGYTVNSHWMSWVRQSPTKGLEWVAEIKLKSNKYASHFAESVKGRFTITRDNSQSTVYLQMNSLRAEDTGIYYCTSKFSDYFDVWGAGSTVTVAS

SEQ ID NO: 31 - protein, 249 aa, synthetic construct
  REGION 1..249: note = Description of sequence: Monoclonal antibody component, particularly, amino acid sequence of a scFv including the VH and the VL of the S1-450-10 anti-FGFR4-Delta mAb
DIVMTQAAPSISVTPGESVSISCRSSESLLHSNGNTYLYWFLQRPGQSPQVLIYRMSSLASGVPDRFSGSGSGTVFILRISRVEAEDVGVYYCMQHLEYPFTFGAGTKLELKGSTSGGGSGGGSGGGGSSEVKLEESGGGLVQPGGSMKLSCVASGFTVNTHWMNWVRQSPEKGLEWVAEIRLKSNNYATHYAESVKGRFTISRDNSQSTVYLQMNNLRTEDTGIYYCTSKYNWYFDVWGAGTTVTVSS

SEQ ID NO: 32 - protein, 244 aa, synthetic construct
  REGION 1..244: note = Description of sequence: Monoclonal antibody component, particularly, amino acid sequence of a scFv including the VH and the VL of the S1-509-7 anti-FGFR4-Delta mAb
DIVMTQSPSSLSASVGDRVTITCRASQDVSGNTYLDWYQQKPGKAPKLLIYRMSRLPSGVPSRFSGSGSGTDFTLTISRLEPEDFAVYYCMQNMEFPFGSGTKLDIKGSTSGGGSGGGSGGGGSSEVKLEESGGGLVQPGGSMKLTCTASGYSVNTHFLNWVRQSPTKGLEWVAEIRLRSNDYATHFAESVKGRFTITRDGSQSTVYLQMNHLRTEDTGIYYCTSRYSDYFDVWGAGSTVTVAS

SEQ ID NO: 33 - protein, 244 aa, synthetic construct
  REGION 1..244: note = Description of sequence: Monoclonal antibody component, particularly, amino acid sequence of a scFv including the VH and the VL of the S1-509-8 anti-FGFR4-Delta mAb
DIVMTQTPASLSASVGDRVTMTCRASQSVSGNTYLNWYQHKPGKAPKLLIFKLSRLTGGVPSRFSGSGSGTDFTLTISRLEPEDFAVYYCMKHLEYKFGQGTKLEIKGSTSGGGSGGGSGGGGSSEVKLEDSGGGLVQPGGSMKLSCIASGFAVNTYWMNWVRQSPTKGLEWVPEIRVKSNGYATHYAEAVKGRFTITRDGSQSTVFLQMNNLRTEDTGIYYCTTKFSDYFDVWGVGTTVTVAS

SEQ ID NO: 34 - protein, 18 aa, synthetic construct
  REGION 1..18: note = Description of sequence: amino acid sequence of an exemplary peptide linker for antibody-based CARs
GSTSGGGSGGGSGGGGSS

SEQ ID NO: 35 - protein, 22 aa, synthetic construct
  REGION 1..22: note = Description of sequence: amino acid sequence of an exemplary signaling peptide
MLLLVTSLLLCELPHPAFLLIP

SEQ ID NO: 36 - protein, 107 aa, synthetic construct
  REGION 1..107: note = Description of sequence: amino acid sequence of exemplary CD28 transmembrane and signaling domains
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 37 - protein, 112 aa, synthetic construct
  REGION 1..112: note = Description of sequence: amino acid sequence of an exemplary CD3? T cell activating domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 19 aa, synthetic construct
  REGION 1..19: note = Description of sequence: nucleotide sequences of FGFR4-Delta Forward Primer
TGGAGGCCTCTGAGGAAGT

SEQ ID NO: 39 - protein, 20 aa, synthetic construct
  REGION 1..20: note = Description of sequence: nucleotide sequences of FGFR4-Delta Reverse Primer
GGGTCCTCATCATCGTTGCT

SEQ ID NO: 40 - protein, 16 aa, synthetic construct
  REGION 1..16: note = Description of sequence: nucleotide sequence of FGFR4-Delta Probe
CAAGGAGTCAAGCTCC

SEQ ID NO: 41 - protein, 714 aa, synthetic construct
  REGION 1..714: note = Description of sequence: the amino acid sequence of FGFR4-Delta
MRLLLALLGVLLSVPGPPVLSLEASEEVELDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 42 - protein, 2145 aa, synthetic construct
  REGION 1..2145: note = Description of sequence: the polynucleotide sequence encoding FGFR4-Delta
ATGCGGCTGCTGCTGGCCCTGTTGGGGGTCCTGCTGAGTGTGCCTGGGCCTCCAGTCTTGTCCCTGGAGGCCTCTGAGGAAGTGGAGCTTGACTCCTTGACCTCCAGCAACGATGATGAGGACCCCAAGTCCCATAGGGACCCCTCGAATAGGCACAGTTACCCCCAGCAAGCACCCTACTGGACACACCCCCAGCGCATGGAGAAGAAACTGCATGCAGTACCTGCGGGGAACACCGTCAAGTTCCGCTGTCCAGCTGCAGGCAACCCCACGCCCACCATCCGCTGGCTTAAGGATGGACAGGCCTTTCATGGGGAGAACCGCATTGGAGGCATTCGGCTGCGCCATCAGCACTGGAGTCTCGTGATGGAGAGCGTGGTGCCCTCGGACCGCGGCACATACACCTGCCTGGTAGAGAACGCTGTGGGCAGCATCCGCTATAACTACCTGCTAGATGTGCTGGAGCGGTCCCCGCACCGGCCCATCCTGCAGGCCGGGCTCCCGGCCAACACCACAGCCGTGGTGGGCAGCGACGTGGAGCTGCTGTGCAAGGTGTACAGCGATGCCCAGCCCCACATCCAGTGGCTGAAGCACATCGTCATCAACGGCAGCAGCTTCGGAGCCGACGGTTTCCCCTATGTGCAAGTCCTAAAGACTGCAGACATCAATAGCTCAGAGGTGGAGGTCCTGTACCTGCGGAACGTGTCAGCCGAGGACGCAGGCGAGTACACCTGCCTCGCAGGCAATTCCATCGGCCTCTCCTACCAGTCTGCCTGGCTCACGGTGCTGCCAGAGGAGGACCCCACATGGACCGCAGCAGCGCCCGAGGCCAGGTATACGGACATCATCCTGTACGCGTCGGGCTCCCTGGCCTTGGCTGTGCTCCTGCTGCTGGCCGGGCTGTATCGAGGGCAGGCGCTCCACGGCCGGCACCCCCGCCCGCCCGCCACTGTGCAGAAGCTCTCCCGCTTCCCTCTGGCCCGACAGTTCTCCCTGGAGTCAGGCTCTTCCGGCAAGTCAAGCTCATCCCTGGTACGAGGCGTGCGTCTCTCCTCCAGCGGCCCCGCCTTGCTCGCCGGCCTCGTGAGTCTAGATCTACCTCTCGACCCACTATGGGAGTTCCCCCGGGACAGGCTGGTGCTTGGGAAGCCCCTAGGCGAGGGCTGCTTTGGCCAGGTAGTACGTGCAGAGGCCTTTGGCATGGACCCTGCCCGGCCTGACCAAGCCAGCACTGTGGCCGTCAAGATGCTCAAAGACAACGCCTCTGACAAGGACCTGGCCGACCTGGTCTCGGAGATGGAGGTGATGAAGCTGATCGGCCGACACAAGAACATCATCAACCTGCTTGGTGTCTGCACCCAGGAAGGGCCCCTGTACGTGATCGTGGAGTGCGCCGCCAAGGGAAACCTGCGGGAGTTCCTGCGGGCCCGGCGCCCCCCAGGCCCCGACCTCAGCCCCGACGGTCCTCGGAGCAGTGAGGGGCCGCTCTCCTTCCCAGTCCTGGTCTCCTGCGCCTACCAGGTGGCCCGAGGCATGCAGTATCTGGAGTCCCGGAAGTGTATCCACCGGGACCTGGCTGCCCGCAATGTGCTGGTGACTGAGGACAATGTGATGAAGATTGCTGACTTTGGGCTGGCCCGCGGCGTCCACCACATTGACTACTATAAGAAAACCAGCAACGGCCGCCTGCCTGTGAAGTGGATGGCGCCCGAGGCCTTGTTTGACCGGGTGTACACACACCAGAGTGACGTGTGGTCTTTTGGGATCCTGCTATGGGAGATCTTCACCCTCGGGGGCTCCCCGTATCCTGGCATCCCGGTGGAGGAGCTGTTCTCGCTGCTGCGGGAGGGACATCGGATGGACCGACCCCCACACTGCCCCCCAGAGCTGTACGGGCTGATGCGTGAGTGCTGGCACGCAGCGCCCTCCCAGAGGCCTACCTTCAAGCAGCTGGTGGAGGCGCTGGACAAGGTCCTGCTGGCCGTCTCTGAGGAGTACCTCGACCTCCGCCTGACCTTCGGACCCTATTCCCCCTCTGGTGGGGACGCCAGCAGCACCTGCTCCTCCAGCGATTCTGTCTTCAGCCACGACCCCCTGCCATTGGGATCCAGCTCCTTCCCCTTCGGGTCTGGGGTGCAGACATGA